(19) United States
(12) Patent Application Publication
(10) Pub. No.: US 2022/0213066 A1
(43) Pub. Date: Jul. 7, 2022

(54) SALTS OF A CLASS OF PYRIMIDINE COMPOUNDS, POLYMORPHS, AND PHARMACEUTICAL COMPOSITIONS THEREOF, PREAPRATION METHODS THEREFOR AND USES THEREOF

(71) Applicants: INVENTISBIO CO., LTD., Shanghai (CN); BETTA PHARMACEUTICALS CO., LTD, Zhejiang (CN)

(72) Inventors: Xing DAI, Shanghai (CN); Yueheng JIANG, Shanghai (CN); Yanqin LIU, Shanghai (CN)

(21) Appl. No.: 17/611,458

(22) PCT Filed: May 15, 2020
(86) PCT No.: PCT/CN2020/090429

§ 371 Date: Nov. 15, 2021

(30) Foreign Application Priority Data
May 16, 2019 (CN) 201910406013.1

(51) Int. Cl. C07D 403/04

(52) U.S. Cl. CPC C07D 403/04; C07B 2200/13

(57) ABSTRACT

The present invention relates to a salt of a Compound 1 and polymorphs thereof, and pharmaceutical compositions containing the same, wherein the salt is preferably hydrochloride, phosphate, tosilate, benzene sulfonate, succinate, sulfate, monohydrobromate, dihydrobromate, etc. The present invention further relates to methods for preparing the described substances, their uses, and pharmaceutical preparations containing these salts and crystalline forms.

### TECHNICAL FIELD

The present invention relates to salts and polymorphs of a pyrimidine compound, and a pharmaceutical composition containing the same, a method for preparing various salts and polymorphs, and their use in preparing a pharmaceutical composition.

### BACKGROUND

Epidermal growth factor receptor (EGFR) is a receptor tyrosine protein kinase, which is a transmembrane protein belonging to the erbB receptor family

EGFR regulates cell proliferation, survival, adhesion, migration and differentiation. It is over-activated or continuously activated in a variety of tumor cells, such as lung cancer, breast cancer, prostate cancer cells and the like. Abnormal activation of EGFR plays a key role in tumor transformation and growth. Blocking the activation of EGFR has been clinically proven to be one of the effective treatment methods for targeting tumor cells. EGFR is expressed in 50% of NSCLC (non-small cell lung cancer) patients. This makes EGFR and its family members the main candidates for targeted therapy. Gefitinib and Erlotinib are the first-generation small molecule EGFR inhibitors, mainly used to treat advanced NSCLC. It has clinically shown that gefitinib or erlotinib is effective for approximately 10% of white NSCLC patients and approximately 35% of Asian NSCLC patients. Analysis results show that most NSCLC patients with EGFR activating mutations have a significantly higher response rate to EGFR-tyrosine kinase inhibitors (TKI) compared with NSCLC patients with wild type EGFR.

However, clinical studies have shown that many patients quickly (12-14 months) develop resistance to these small molecule EGFR inhibitors, that is, acquired drug resistance. The gatekeeper residue T790M mutation is a mutation point in the exon 20 of EGFR, and it is one of the main mechanisms causing drug resistance. It has achieved great success by recent research on a new generation of inhibitors against these EGFR mutations. Afatinib is a potent and irreversible dual inhibitor of EGFR and human epidermal growth factor receptor 2 (HER2) tyrosine kinase. Other highly active, irreversible inhibitors with similar multi targets, such as Canertinib, Dacomitinib are also in late-stage clinical trials. These new second-generation irreversible inhibitors have a potent inhibitory effect on EGFR L858R and T790M mutations, and have significant effects on cancer patients who are already resistant to gefitinib or erlotinib. However, these second-generation inhibitors of EGFR mutant also have potent inhibitory effect on wild-type EGFR (WT-EGFR). Clinical studies have proven that the inhibition on wild-type EGFR can cause drug toxicity and side effects in most patients, for example, some patients encounter skin rash or diarrhea.

To overcome the toxicity and side effects of these second-generation EGFR inhibitors, it is necessary to reduce the inhibitory effect on wild-type EGFR (WT-EGFR). The new generation of EGFR inhibitors should maintain strong inhibition on EGFR L858R activating mutant, Exon19 deletion activating mutant and T790M resistance mutant, while having relatively weak inhibitory effect on WT-EGFR and other tyrosine protein kinase receptors. Without concerns on the side effects of second-generation EGFR mutant inhibitors such as afatinib, this kind of compounds can be used for the treatment of cancer patients with EGFR L858R activating mutant and Exon19 deletion activating mutant, and for the treatment of cancer patients with EGFR-T790M mutant who are resistant to the first generation of EGFR inhibitors such as gefitinib, erlotinib or icotinib.

Chinese patent application CN105085489A relates to a class of pyrimidine or pyridine compounds, and their pharmaceutically acceptable salts, stereoisomers, prodrugs and solvates, their preparation methods, pharmaceutical compositions and medical uses. This application shows many pyrimidine or pyridine compounds having high inhibitory activity against EGFR mutants (one or more mutants, such as EGFR L858R activating mutant, Exon19 deletion activating mutant and/or T790M resistance mutant), while having relatively low inhibitory activity against wild-type EGFR.

Compound 1 (see Example 104 of CN105085489A) as shown below, the compound described in CN105085489A, has good biological activity and safe toxicity parameters. This class of compounds has a good function in the treatment of cancers with EGFR activating mutants and/or EGFR drug-resistant mutations. CN105085489A describes the synthesis of Compound 1 and methanesulfonate thereof. In order to further improve the physicochemical properties of Compound 1, such as stability, hygroscopicity, solubility, etc., which may be beneficial to its production, preparation, synthesis, and/or pharmaceutical applications, the present inventor has developed a novel salt form and a polymorphism of Compound 1 after conducting in-depth research.

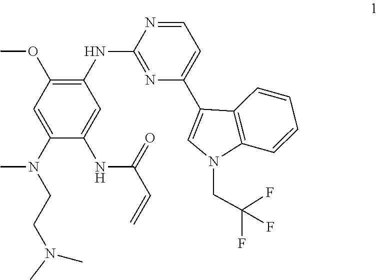 [SMILES: C=CC(=O)Nc1cc(Nc2nccc(-c3cn(CC(F)(F)F)c4ccccc34)n2)c(OC)cc1N(C)CCN(C)C]

### DESCRIPTION

One of the objects of the present invention is to provide a salt form of a pyrimidine Compound 1, preferably its p-toluenesulfonate, benzenesulfonate, succinate, hydrochloride, phosphate, sulfate, or hydrobromide, for example, a salt form and/or crystalline form thereof prepared in Examples 1-9.

The Compound 1 described herein refers to a compound with the following structure:

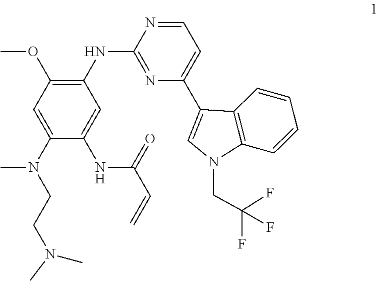 [SMILES: C=CC(=O)Nc1cc(Nc2nccc(-c3cn(CC(F)(F)F)c4ccccc34)n2)c(OC)cc1N(C)CCN(C)C]

The “salts” described herein include pharmaceutically acceptable salts as well as pharmaceutically unacceptable salts. It is not preferable to apply the pharmaceutically unacceptable salts to patients, but these salts can be used to provide pharmaceutical intermediates and bulk pharmaceutical forms.

Compound 1 can form a salt with one or two equivalents of acid (abbreviated as mono-salt or di-salt), for example, its hydrobromide can be monohydrobromide or dihydrobromide. Generally, when preparing a salt form of Compound 1, the corresponding mono- or di-salt can be generated by controlling the molar ratio of the compound to the corresponding acid. However, it is difficult to completely control the equivalent of 1:1 or 1:2 during actual operation, and in large-scale preparations, due to the locally excessive presence of acid or Compound 1, a mixture of a mono-salt and a di-salt may be formed. Because the physical and chemical properties of a mono-salt are different from those of a di-salt, the formation of this mixture will result in non-uniform properties of the final product. Therefore, it will bring great convenience to the preparation and production if the formation of a certain salt type is relatively easily controlled, and final products with uniform qualities can be obtained more easily. The inventor has discovered by accident that for p-toluenesulfonate, benzenesulfonate, succinate, hydrochloride, phosphate, and sulfate of Compound 1, a mono-salt can be formed in high yields at a molar ratio of the compound to the corresponding acid of slightly less than 1:1, such as 1:1.1 (acid excess), so the scale-up process is simplified and the efficiency is improved.

As described herein, compared with Compound 1, some salt forms of Compound 1, such as hydrochloride, phosphate, p-toluenesulfonate, benzenesulfonate, succinate, sulfate, hydrobromide (including monohydrobromide or dihydrobromide), have more or less improved water solubility, and some polymorphs of these salt forms (especially p-toluenesulfonate crystalline form I, benzenesulfonate crystalline form I, phosphate crystalline form I, etc.) have properties such as high stability, low moisture absorption, which is beneficial to the production and preparation of Compound 1, and is of great significance to its final marketization.

In some embodiments, the present invention provides a p-toluenesulfonate of Compound 1, preferably a crystalline form I of p-toluenesulfonate of Compound 1. In the present application, the crystalline form I of p-toluenesulfonate of Compound 1 refers to a crystalline form with one or more of the following characteristics: 1) its X-ray powder diffraction pattern has diffraction peaks at least at one or more positions (1, 2, 3, 4, 5, or 6) of 7.22, 7.90, 9.30, 10.46, 14.64, 15.36, ±0.2° 2θ; 2) its DSC graph has an endothermic peak with an onset temperature of 161.54° C.±5° C. In the crystalline form I of p-toluenesulfonate of Compound 1, the molar ratio of Compound 1 to p-toluenesulfonic acid is about 1:1. In some embodiments, the X-ray powder diffraction pattern of the crystalline form I of p-toluenesulfonate of Compound 1 has 6 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 7.221 | 13.478 | 17.536 | 20.498 | 23.679 | 28.449 |
| 7.904 | 14.638 | 18.385 | 21.368 | 24.457 | 29.728 |
| 9.293 | 15.36 | 19.004 | 22.224 | 25.408 | 30.176 |
| 10.459 | 15.708 | 19.25 | 22.529 | 26.66 | 31.107 |
| 12.015 | 16.892 | 20.231 | 23.184 | 27.37 | |

In some embodiments, the X-ray powder diffraction pattern of the crystalline form I of p-toluenesulfonate of Compound 1 has the main peaks in FIG. 14, that is, having peaks at the corresponding 2θ angle ±0.2°, however, the intensities of the peaks might be different from those in FIG. 14. The main peak of the X-ray powder diffraction pattern herein means a peak in the X-ray powder diffraction pattern with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of p-toluenesulfonate of Compound 1 is substantially the same as that in FIG. 14. The substantially the same X-ray powder diffraction pattern means that the 2θ angles of the diffraction peaks in two patterns are substantially the same within the experimental error range, however, the intensities of the peaks might be different. Preferably, the DSC graph of the crystalline form is also substantially the same as that in FIG. 15. The substantially the same DSC graph means that the endothermic peaks in two graphs, such as their starting temperatures, are substantially the same within the experimental error range.

In some embodiments, the present invention provides a crystalline form I of p-toluenesulfonate of Compound 1 with high purity, for example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form I of its p-toluenesulfonate.

The crystalline form I of p-toluenesulfonate of Compound 1 can be usually obtained by the following method: Compound 1 and p-toluenesulfonic acid are mixed in a suitable solvent at a molar ratio of about 1:1, and then the p-toluenesulfonate salt of Compound 1 crystalizes. In some embodiments, the molar ratio of Compound 1 to p-toluenesulfonic acid may be slightly less than 1:1 (acid excess), for example, about 1:1.1; about 1:1.15; about 1:1.2. The solvent can be one or more organic solvents, such as acetone. In some embodiments, both the salt-forming reaction and crystallization can be carried out under stirring at room temperature. A typical method for preparing the crystalline form I of p-toluenesulfonate of Compound 1 is described in details in example 3.

The crystalline form I of p-toluenesulfonate of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its p-toluenesulfonate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a benzenesulfonate of Compound 1, preferably a crystalline form I of benzenesulfonate of Compound 1. As used herein, the crystalline form I of benzenesulfonate of Compound 1 refers to a crystalline form with one or more of the following characteristics: 1) its X-ray powder diffraction pattern has diffraction peaks at least at one or more positions (1, 2, 3, 4, or 5, preferably 5) of 8.41, 16.53, 18.78, 21.18, 23.16, ±0.2°, 2θ; 2) its DSC graph has an endothermic peak with an onset temperature of 155.49° C.±5° C. In the crystalline form I of benzenesulfonate of Compound 1, the molar ratio of Compound 1 to benzenesulfonic acid is about 1:1. In some embodiments, the X-ray powder diffraction pattern of the crystalline form I of benzenesulfonate of Compound 1 has 6 or more (such as 10, 16, or 20) X-ray diffraction peaks as shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 7.675 | 13.3 | 17.122 | 21.177 | 24.769 | 30.277 |
| 8.411 | 14.595 | 17.728 | 21.532 | 25.162 | 33.549 |
| 10.009 | 15.523 | 18.196 | 22.191 | 25.846 | 34.355 |
| 10.494 | 15.89 | 18.782 | 23.163 | 26.396 | 34.441 |
| 10.766 | 16.534 | 19.181 | 24.082 | 27.523 | 39.824 |
| 11.143 | 16.845 | 20.084 | 24.415 | 29.625 | |

In some preferred embodiments, the X-ray powder diffraction pattern of the crystalline form I of benzenesulfonate of Compound 1 has diffraction peaks at 7.68, 8.41, 14.60, 15.52, 16.53, 16.85, 17.73, 18.78, 20.08, 21.18, 23.16, 24.42, and 24.76, ±0.2° 2θ.

In some embodiments, the X-ray powder diffraction pattern of the crystalline form I of benzenesulfonate of Compound 1 has the main peaks in FIG. 19, that is, having peaks at the corresponding 2θ angle ±0.2°, however, the intensities of the peaks might be different from those shown in FIG. 19, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of benzenesulfonate of Compound 1 is substantially the same as that in FIG. 19. Preferably, the DSC graph of the crystalline form is also substantially the same as that in FIG. 20.

In some embodiments, the present invention provides a crystalline form I of benzenesulfonate of Compound 1 with high purity. For example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form I of its benzenesulfonate.

The crystalline form I of benzenesulfonate of Compound 1 can usually be obtained by the following method: Compound 1 and p-benzenesulfonic acid are mixed in an appropriate solvent at a molar ratio of about 1:1, and then the form I of benzenesulfonate of Compound 1 crystallizes. In some embodiments, the molar ratio of Compound 1 to benzenesulfonic acid may be slightly less than 1:1 (acid excess), for example, about 1:1.1; about 1:1.15; about 1:1.2. The solvent can be one or more organic solvents, such as acetone, acetonitrile. In some embodiments, both the salt-forming reaction and crystallization can be carried out under stirring at room temperature. In some embodiments, the solvent used in the salt-forming reaction may be different from that used in the crystallization. A typical method for preparing the crystalline form I of benzenesulfonate of Compound 1 is described in details in example 4.

The crystalline form I of benzenesulfonate of Compound 1 can usually be combined with a pharmaceutically acceptable carrier or diluent to form a pharmaceutical composition. Preferably, Compound 1 is predominantly present (for example, in about 80wt %, about 90wt %, about 95wt %, or more, or other forms of Compound 1 that cannot be detected by XRPD) in the pharmaceutical composition in the form of crystalline form I of its benzenesulfonate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a succinate of Compound 1, preferably a crystalline form I of succinate of Compound 1. As used herein, the crystalline form I of succinate of Compound 1 refers to a crystalline form with one or more of the following characteristics: 1) its X-ray powder diffraction pattern has diffraction peaks at least at one or more positions (1, 2, 3, 4, or 5, preferably 5) of 7.38, 10.21, 11.59, 17.55, 23.38, ±0.2° 2θ; 2) its DSC graph has an endothermic peak with an onset temperature of 108.3° C.±5° C. In the crystalline form I of succinate of Compound 1, the molar ratio of Compound 1 to succinic acid is about 1:1. In some embodiments, the X-ray powder diffraction pattern of the crystalline form I of succinate of Compound 1 has 6 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.946 | 13.549 | 17.811 | 21.142 | 25.463 | 29.9 |
| 7.376 | 13.952 | 18.449 | 21.864 | 25.892 | 30.547 |
| 9.175 | 14.89 | 18.642 | 22.144 | 26.463 | 31.357 |
| 9.674 | 15.942 | 19.051 | 23.376 | 27.119 | 31.958 |
| 10.209 | 16.57 | 19.42 | 24.111 | 27.829 | 33.223 |
| 10.672 | 16.859 | 19.595 | 24.402 | 28.567 | 35.668 |
| 11.594 | 17.554 | 20.418 | 24.975 | 29.326 | 36.201 |

In some preferred embodiments, the X-ray powder diffraction pattern of the crystalline form I of succinate of Compound 1 has diffraction peaks at 7.38, 9.18, 9.67, 10.21, 10.67, 11.59, 13.55, 14.89, 16.86, 17.55, 19.05, 19.42, 19.60, 23.38, 24.11, 24.40, 27.83, 29.90, and 30.55, ±0.2° 2θ.

In some embodiments, the X-ray powder diffraction pattern of the crystalline form I of succinate of Compound 1 has the main peaks in FIG. 24, that is, having peaks at the corresponding 2θ angle ±0.2°, however, the intensities of the peaks might be different from those shown in FIG. 24, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of succinate of Compound 1 is substantially the same as that in FIG. 24. Preferably, the DSC graph of the crystalline form is also substantially the same as that in FIG. 25.

In some embodiments, the present invention provides a crystalline form I of succinate of Compound 1 with high purity. For example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form I of its succinate.

The crystalline form I of succinate of Compound 1 can usually be obtained by the following method: Compound 1 and succinic acid are mixed in a suitable solvent at a molar ratio of about 1:1, and then the form I of succinate of Compound 1 crystallizes. In some embodiments, the molar ratio of Compound 1 to succinic acid may be slightly less than 1:1 (acid excess), for example, about 1:1.1; about 1:1.15; about 1:1.2. The solvent can be one or more organic solvents, such as acetone and acetonitrile. In some embodiments, both the salt-forming reaction and crystallization can be carried out under stirring at room temperature. In some embodiments, the solvent used in the salt-forming reaction may be different from that used in the crystallization. A typical method for preparing the crystalline form I of succinate of Compound 1 is described in details in Example 5.

The crystalline form I of succinate of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its succinate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a crystalline form II of succinate of Compound 1. As used herein, the crystalline form II of succinate of Compound 1 refers to a crystalline form with one or more of the following characteristics: 1) its X-ray powder diffraction pattern has diffraction peaks at least at one or more positions (1, 2, 3, 4, 5, 6, 7, or 8, preferably 5 or more, more preferably, 8) of 7.32, 9.02, 9.65, 10.09, 11.63, 17.53, 19.47, 23.45, ±0.2° 2θ; 2) its DSC graph has an endothermic peak with an onset temperature of 139.9° C.±5° C. In the crystalline form II of succinate of Compound 1, the molar ratio of Compound 1 to succinic acid is about 1:1. In some embodiments, the X-ray powder diffraction pattern of the crystalline form II of succinate of Compound 1 has 8 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.89 | 13.881 | 18.644 | 22.561 | 25.942 | 30.688 |
| 7.321 | 14.734 | 18.945 | 23.148 | 26.482 | 31.826 |
| 8.014 | 15.781 | 19.474 | 23.454 | 26.897 | 33.307 |
| 9.022 | 16.446 | 19.702 | 23.786 | 27.402 | 34.561 |
| 9.652 | 16.774 | 20.376 | 24.171 | 28.108 | 35.276 |
| 10.087 | 17.534 | 21.106 | 24.428 | 29.431 | 36.167 |
| 10.51 | 17.821 | 21.8 | 24.839 | 29.892 | 36.427 |
| 11.63 | 18.131 | 22.293 | 25.349 | 30.33 | 39.608 |
| 13.604 | | | | | |

In some preferred embodiments, the X-ray powder diffraction pattern of the crystalline form II of succinate of Compound 1 has diffraction peaks at 7.32, 9.02, 9.65, 10.09, 10.51, 11.63, 13.60, 14.73, 16.45, 16.77, 17.53, 18.13, 19.47, 19.70, 23.45, 23.79, and 24.43, ±0.2° 2θ.

In some embodiments, the X-ray powder diffraction pattern of the crystalline form II of succinate of Compound 1 has the main peaks in FIG. 29, that is, having peaks at the corresponding 2θ angle ±0.2, however, the intensities of the peaks might be different from those shown in FIG. 29, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form II of succinate of Compound 1 is substantially the same as that in FIG. 29. Preferably, the DSC graph of the crystalline form is also substantially the same as that in FIG. 30.

In some embodiments, the present invention provides a crystalline form II of succinate of Compound 1 with high purity. For example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form II of its succinate.

The crystalline form II of succinate of Compound 1 can usually be obtained by the following method: Compound 1 and succinic acid are mixed in a suitable solvent at a molar ratio of about 1:1, and then the form II of succinate of Compound 1 crystallizes. In some embodiments, the molar ratio of Compound 1 to succinic acid may be slightly less than 1:1 (acid excess), for example, about 1:1.1; about 1:1.15; about 1:1.2. The solvent can be one or more organic solvents, such as ethyl acetate, 2-butanone. In some embodiments, both the salt-forming reaction and crystallization can be carried out under stirring at room temperature. In some embodiments, the solvent used in the salt-forming reaction may be different from that used in the crystallization. A typical method for preparing the crystalline form II of succinate of Compound 1 is described in details in Example 6.

The crystalline form II of succinate of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form II of its succinate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a hydrochloride of Compound 1, preferably a crystalline form III of hydrochloride of Compound 1. As used herein, the crystalline form III of hydrochloride of Compound 1 refers to a crystalline form with one or more of the following characteristics: 1) its X-ray powder diffraction pattern has diffraction peaks at least at one or more positions (1, 2, 3, 4, 5, 6, 7, or 8, preferably 5 or more, more preferably, 8) of 6.39, 7.35, 10.03, 11.48, 15.27, 21.04, 21.87, 23.35, 24.94, ±0.2° 2θ; 2) its DSC graph has an endothermic peak with an onset temperature of 270.75° C.±5° C. In the crystalline form III of hydrochloride of Compound 1, the molar ratio of Compound 1 to hydrochloric acid is about 1:1. In some embodiments, the X-ray powder diffraction pattern of the crystalline form III of hydrochloride of Compound 1 has 8 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.385 | 13.255 | 19.4 | 22.134 | 26.206 | 29.921 |
| 7.353 | 14.632 | 20.042 | 22.745 | 26.789 | 31.559 |
| 7.872 | 15.266 | 20.313 | 23.353 | 27.255 | 32.794 |
| 10.033 | 15.657 | 20.694 | 23.621 | 27.481 | 33.388 |
| 11.483 | 16.947 | 21.037 | 24.101 | 27.875 | 37.271 |
| 12.445 | 18.181 | 21.485 | 24.944 | 28.937 | 39.086 |
| 12.977 | 18.713 | 21.867 | | | |

In some preferred embodiments, the X-ray powder diffraction pattern of the crystalline form III of hydrochloride of Compound 1 has diffraction peaks at 6.39, 7.35, 7.87, 10.03, 11.48, 15.27, 21.04, 21.87, 22.13, 22.74, 23.35, 24.94 and 26.79, ±0.2° 2θ.

In some embodiments, the X-ray powder diffraction pattern of the crystalline form III of hydrochloride of Compound 1 has the main peaks in FIG. 4, that is, having peaks at the corresponding 2θ angle ±0.2°, however, the intensities of the peaks might be different from those shown in FIG. 4, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form III of hydrochloride of Compound 1 is substantially the same as that in FIG. 4. Preferably, the DSC graph of the crystalline form is also substantially the same as that in FIG. 5.

In some embodiments, the present invention provides a crystalline form III of hydrochloride of Compound 1 with high purity, for example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form III of its hydrochloride.

The crystalline form III of hydrochloride of Compound 1 can usually be obtained by the following method: Compound 1 and hydrochloric acid are mixed in a suitable solvent at a molar ratio of about 1:1, and then the form III of hydrochloride of Compound 1 crystallizes. In some embodiments, the molar ratio of Compound 1 to hydrochloric acid may be slightly less than 1:1 (acid excess), for example, about 1:1.1; about 1:1.15; about 1:1.2. The solvent can be one or more organic solvents, such as acetonitrile and dichloromethane. In some embodiments, both the salt-forming reaction and crystallization can be carried out under stirring at room temperature. In some embodiments, the solvent used in the salt-forming reaction may be different from that used in the crystallization. A typical method for preparing the crystalline form III of hydrochloride of Compound 1 is described in details in Example 1.

The crystalline form III of hydrochloride of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form III of its hydrochloride. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a phosphate of Compound 1, preferably a crystalline form I of phosphate of Compound 1. As used herein, the crystalline form I of phosphate of Compound 1 refers to a crystalline form with one or more of the following characteristics: 1) its X-ray powder diffraction pattern has diffraction peaks at least at one or two positions (preferably 2) of 8.14, 16.32, ±0.2° 2θ; 2) its DSC graph has an endothermic peak with an onset temperature of 234.95° C.±5° C. In the crystalline form I of phosphate of Compound 1, the molar ratio of Compound 1 to phosphoric acid is about 1:1. In some embodiments, the X-ray powder diffraction pattern of the crystalline form I of phosphate of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 8.144 | 13.554 | 17.395 | 20.994 | 24.015 | 29.882 |
| 8.573 | 14.334 | 17.752 | 21.366 | 24.715 | 31.536 |
| 9.48 | 14.767 | 18.48 | 22.361 | 26.218 | 32.976 |
| 10.988 | 15.671 | 19.362 | 22.992 | 26.91 | 37.285 |
| 12.698 | 16.316 | 20.389 | 23.451 | 29.013 | 39.543 |

In some preferred embodiments, the X-ray powder diffraction pattern of the crystalline form I of phosphate of Compound 1 has diffraction peaks at 8.14, 16.32, 17.75 and 20.99, ±0.2° 2θ.

In some embodiments, the X-ray powder diffraction pattern of the crystalline form I of phosphate of Compound 1 has the main peaks in FIG. 9, that is, having peaks at the corresponding 2θangle ±0.2°, however, the intensities of the peaks might be different from those shown in FIG. 9, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of phosphate of Compound 1 is substantially the same as that in FIG. 9. Preferably, the DSC graph of the crystalline form is also substantially the same as that in FIG. 10.

In some embodiments, the present invention provides a crystalline form I of phosphate of Compound 1 with high purity. For example, in some embodiments, Compound 1 is predominantly present (for example, in about 80 wt %, about 90 wt %, about 95 wt %, or more, or other forms of Compound 1 that cannot be detected by XRPD) in high-purity substance in the form of crystalline form I of its phosphate.

The crystalline form I of phosphate of Compound 1 can usually be obtained by the following method: Compound 1 and phosphoric acid are mixed in a suitable solvent at a molar ratio of about 1:1, and then the form I of phosphate of Compound 1 crystallizes. In some embodiments, the molar ratio of Compound 1 to phosphoric acid can be slightly less than 1:1 (acid excess), for example, about 1:1.1; about 1:1.15; about 1:1.2. The solvents can be one or more organic solvent, such as acetone. In some embodiments, both the salt-forming reaction and crystallization can be carried out under stirring at room temperature. In some embodiments, the solvent used in the salt-forming reaction may be different from that used in the crystallization. A typical method for preparing the crystalline form I of phosphate of Compound 1 is described in details in Example 2.

The crystalline form I of phosphate of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its phosphate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a sulfate of Compound 1, preferably a crystalline form I of sulfate of Compound 1. As used herein, the crystalline form I of sulfate of Compound 1 refers to a crystalline form having one or more of the following characteristics: 1) its X-ray powder diffraction pattern has diffraction peaks at least at one or more (preferably 2 or 3) 10.28, 18.34, 20.64, ±0.2° 2θ; 2) its DSC graph has an endothermic peak with an onset temperature of 255.89° C.±5° C. In the crystalline form I of sulfate of Compound 1, the molar ratio of Compound 1 to sulfuric acid is about 1:1. In some embodiments, the X-ray powder diffraction pattern of the crystalline form I of sulfate of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 9.039 | 14.239 | 20.141 | 21.943 | 27.196 | 31.141 |
| 9.49 | 15.432 | 20.411 | 22.45 | 28.534 | 32.097 |
| 10.275 | 18.342 | 20.635 | 22.792 | 30.647 | 33.216 |
| 11.809 | 19.085 | 21.261 | 24.479 | | |

In some preferred embodiments, the X-ray powder diffraction pattern of the crystalline form I of sulfate of Compound 1 has diffraction peaks at 9.04, 10.28, 18.34, 20.41, 20.64, 27.20 and 28.53, ±0.2° 2θ.

In some embodiments, the X-ray powder diffraction pattern of the crystalline form I of sulfate of Compound 1 has the main peaks in FIG. 32, that is, having peaks at the corresponding 2θ angle ±0.2°, however, the intensities of the peaks might be different from those shown in FIG. 32, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of sulfate of Compound 1 is substantially the same as that in FIG. 32. Preferably, the DSC graph of the crystalline form is also substantially the same as that in FIG. 33.

In some embodiments, the present invention provides a crystalline form I of sulfate of Compound 1 with high purity, for example, in some embodiments, Compound 1 is predominantly present (for example, in about 80 wt %, about 90 wt %, about 95 wt %, or more, or other forms of Compound 1 that cannot be detected by XRPD) in the high-purity substance in the form of crystalline form I of its sulfate.

The crystalline form I of sulfate of Compound 1 can usually be obtained by the following method: Compound 1 and sulfuric acid are mixed in a suitable solvent at a molar ratio of about 1:1, and then the form I of sulfate of Compound 1 crystallizes. In some embodiments, the molar ratio of Compound 1 to sulfuric acid may be slightly less than 1:1 (acid excess), for example, about 1:1.1; about 1:1.15; about 1:1.2. The solvent can be one or more organic solvents, such as ethyl acetate. In some embodiments, both the salt-forming reaction and crystallization can be carried out under stirring at room temperature. In some embodiments, the solvent used in the salt-forming reaction may be different from that used in the crystallization. A typical method for preparing the crystalline form I of sulfate of Compound 1 is described in details in Example 7.

The crystalline form I of sulfate of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its sulfate. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a hydrobromide of Compound 1, such as a crystalline form I of monohydrobromide of Compound 1. As used herein, the crystalline form I of monohydrobromide of Compound 1 refers to a crystalline form with one or more of the following characteristics: 1) its X-ray powder diffraction pattern has diffraction peaks at least at one or two positions of 6.10, 24.73 ±0.2° 2θ; 2) its DSC graph has two endothermic peaks. In the crystalline form I of monohydrobromide of Compound 1, the molar ratio of Compound 1 to hydrobromic acid is about 1:1. In some embodiments, the X-ray powder diffraction pattern of the crystalline form I of monohydrobromide of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 3.67 | 13.07 | 17.703 | 23.634 | 28.981 | 31.923 |
| 6.104 | 14.58 | 19.27 | 24.73 | 29.532 | 37.951 |
| 10.262 | 15.651 | 20.057 | 26.032 | 30.584 | 39.358 |
| 12.251 | 16.739 | 21.916 | 26.437 | 31.816 | |

In some preferred embodiments, the X-ray powder diffraction pattern of the crystalline form I of monohydrobromide of Compound 1 has diffraction peaks at 6.10, 12.25, 13.07, 14.58, 15.65, 16.74, 19.27, 20.06, 21.92, 24.73, 26.03 and 26.44, ±0.2° 2θ.

In some embodiments, the X-ray powder diffraction pattern of the crystalline form I of monohydrobromide of Compound 1 has the main peaks in FIG. 37, that is, having peaks at the corresponding 2θ angle ±0.2°, however, the intensities of the peaks might be different from those shown in FIG. 37, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of monohydrobromide of Compound 1 is substantially the same as that in FIG. 37. Preferably, the DSC graph of the crystalline form is also substantially the same as that in FIG. 38.

In some embodiments, the present invention provides a crystalline form I of monohydrobromide of Compound 1 with high purity, for example, in some embodiments, Compound 1 is predominantly present (e.g., in about 80 wt %, about 90 wt %, about 95 wt %, or more, or other forms of Compound 1 not detectable by XRPD) in the high-purity substance in the form of crystalline form I of its monohydrobromide.

The crystalline form I of monohydrobromide of Compound 1 can usually be obtained by the following method: Compound 1 and hydrobromic acid are mixed in a suitable solvent at a molar ratio of about 1:1, and then the form I of monohydrobromide of Compound 1 crystallizes. In some embodiments, the molar ratio of Compound 1 to hydrobromic acid may be slightly less than 1:1 (acid excess), for example, about 1:1.1; about 1:1.15; about 1:1.2. The solvent can be one or more organic solvents, such as acetone. In some embodiments, both the salt-forming reaction and crystallization can be carried out under stirring at room temperature. In some embodiments, the solvent used for the salt formation reaction may be different from that used in the crystallization. A typical method for preparing the crystalline form I of monohydrobromide of Compound 1 is described in details in Example 8.

The crystalline form I of monohydrobromide of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its monohydrobromide. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a crystalline form I of dihydrobromide of Compound 1. As used herein, the crystalline form I of dihydrobromide of Compound 1 refers to a crystalline form with one or more of the following characteristics: 1) its X-ray powder diffraction pattern has diffraction peaks at least at one or more positions (such as 1, 2, 3, or 4) of 6.28, 13.12, 19.30, 25.34, ±0.2° 2θ; 2) its DSC graph has two endothermic peaks with onset temperatures at 193.38° C.±5° C. and 230.24° C.±5° C. respectively. In the crystalline form I of dihydrobromide of Compound 1, the molar ratio of Compound 1 to hydrobromic acid is about 1:2. In some embodiments, the X-ray powder diffraction pattern of the crystalline form I of dihydrobromide of Compound 1 has 6 or more (such as 8, 12, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.276 | 12.071 | 18.953 | 22.87 | 28.58 | 33.849 |
| 7.329 | 12.603 | 19.305 | 23.626 | 29.384 | 34.543 |
| 7.771 | 13.122 | 19.605 | 24.148 | 30.618 | 35.211 |
| 9.38 | 14.575 | 20.387 | 25.341 | 31.164 | 36.629 |
| 9.69 | 16.777 | 20.662 | 25.61 | 31.832 | 38.6 |
| 10.493 | 17.067 | 21.148 | 26.424 | 32.348 | 39.414 |
| 11.591 | 18.236 | 21.954 | 27.78 | 33.126 | |

In some preferred embodiments, the X-ray powder diffraction pattern of the crystalline form I of dihydrobromide of Compound 1 has diffraction peaks at 6.28, 13.12, 16.78, 18.95, 19.30, 21.95, 23.63, 25.34, 25.61 and 26.42, ±0.2° 2θ.

In some embodiments, the X-ray powder diffraction pattern of the crystalline form I of dihydrobromide of Compound 1 has the main peaks in FIG. 40, that is, having peaks at the corresponding 2θ angle ±0.2°, however, the intensities of the peaks might be different from those shown in FIG. 40, for example, a peak with a relative intensity of 20% or more, for example, a peak with a relative intensity of 30% or more, 40% or more, 50% or more, 60% or more, 80% or more, 90% or more, or 100%, preferably 30% or more, more preferably 50% or more.

In some embodiments, the X-ray powder diffraction pattern of the crystalline form I of dihydrobromide of Compound 1 is substantially the same as that in FIG. 40. Preferably, the DSC graph of the crystalline form is also substantially the same as that in FIG. 41.

In some embodiments, the present invention provides a crystalline form I of dihydrobromide of Compound 1 with high-purity, for example, in some embodiments, Compound 1 is predominantly present (for example, in about 80 wt %, about 90 wt %, about 95 wt %, or more, or other forms of Compound 1 not detectable by XRPD) in the high-purity substance in the form of crystalline form I of its dihydrobromide.

The crystalline form I of dihydrobromide of Compound 1 can usually be obtained by the following method: Compound 1 and hydrobromic acid are mixed in a suitable solvent at a molar ratio of about 1:2, and then the form I of dihydrobromide of Compound 1 crystallizes. The solvent can be one or more organic solvents, such as acetone and acetonitrile. In some embodiments, both the salt-forming reaction and crystallization can be carried out under stirring at room temperature. In some embodiments, the solvent used for the salt formation reaction may be different from that used in the crystallization. A typical method for preparing the crystalline form I of dihydrobromide of Compound 1 is described in details in Example 9.

The crystalline form I of dihydrobromide of Compound 1 can usually be combined with a pharmaceutically acceptable carrier or diluent to form a pharmaceutical composition. Preferably, Compound 1 is predominantly present (for example, in about 80 wt %, about 90 wt %, about 95 wt %, or more, or other forms of Compound 1 that cannot be detected by XRPD) in the pharmaceutical composition in the form of crystalline form I of its dihydrobromide. In some cases, Compound 1 is the sole active substance in the pharmaceutical composition. In some cases, the pharmaceutical composition contains a therapeutically or preventively effective amount of Compound 1, for example, for non-small cell lung cancer or other EGFR-mediated disorders or diseases described herein.

In some embodiments, the present invention provides a pharmaceutical composition comprising any one or more of the salt forms or crystalline forms described herein and a pharmaceutically acceptable carrier or diluent. Excipients, binders, lubricants, disintegrating agents, coloring agents, flavoring agents, emulsifiers, surfactants, solubilizers, suspending agents, isotonic agents, buffers, preservatives, antioxidants, stabilizers, absorption promoters, etc. which are commonly used in the medical field can also be used in appropriate combinations as needed.

The pharmaceutical composition of the present invention can be in any available dosage form, for example, tablets, capsules and the like. In the case of preparing a tablet-type solid composition, the main active ingredient component can be mixed with a pharmaceutical carrier, such as starch, lactose, magnesium stearate, etc., and the tablet can be coated with sugar or other suitable substances, or it is processed so that the tablet has a prolonged or delayed releasing effect and the tablet releases a predetermined amount of active ingredient in a continuous manner. In the case of preparing a capsule-type solid composition, a capsule can be obtained by mixing the active ingredient with a diluent, and filling the resulting mixture into capsules. In some embodiments, the pharmaceutical composition of the present invention can also be in other dosage forms, such as granules, powders, or syrups and the like which are administered orally, or injections, powder injections, sprays, or suppositories and the like, which are non-orally administered. These preparations can be prepared by conventional methods.

In some embodiments, the salt, crystalline form, and/or pharmaceutical composition of Compound 1 of the present invention can be used for preparing a drug for the treatment or prevention a disorder or disease mediated by activating or resistant mutant form of EGFR, for example, mediated by L858R activating mutant, Exon19 deletion activating mutant and/or T790M resistance mutant of EGFR. In some embodiments, the disorder or disease is cancer. In some embodiments, the disorder or disease includes, but is not limited to: ovarian cancer, cervical cancer, colorectal cancer (e.g., colon adenocarcinoma), breast cancer, pancreatic cancer, glioma, glioblastoma, melanoma, prostate cancer, leukemia, lymphoma, non-Hodgkin's lymphoma, gastric cancer, lung cancer (for example, non-small cell lung cancer), hepatocellular carcinoma, gastrointestinal stromal tumor (GIST), thyroid cancer, cholangiocarcinoma, intrauterine membrane cancer, kidney cancer, anaplastic large cell lymphoma, acute myeloid leukemia (AML), multiple myeloma or mesothelioma.

In the present invention, the activating mutant or resistant mutant form of EGFR may be, for example, L858R activating mutant, Exon19 deletion activating mutant and/or T790M resistance mutant. Therefore, the disorder or disease mediated by the activating mutant or resistant mutant form of EGFR may be, for example, a disorder or disease mediated by L858R activating mutant, Exon19 deletion activating mutant and/or T790M resistance mutant.

The salt, crystalline form, and/or pharmaceutical composition of Compound 1 of the present invention can be specifically used in the prevention or treatment of diseases mediated by the activating mutant or resistant mutant form of EGFR, for example, in the prevention or treatment of diseases, disorders or conditions mediated by L858R activating mutant, Exon19 deletion activating mutant and/or T790M resistance mutant, for example, it can be used in the prevention or treatment in cancer patients who have been resistant to gefitinib, erlotinib, or ectinib.

In another aspect of the present invention, it provides a combined treatment method for cancer, comprising administering a therapeutically effective amount of the salt, crystalline form of Compound 1, and/or pharmaceutical composition thereof of the present invention to an individual in need thereof, with the combination of conventional surgery or radiotherapy or chemotherapy or immuno-tumor therapy. The chemotherapy or immuno-tumor therapy may be administered together, simultaneously, sequentially, or separately with the application of the salt, crystalline form, and/or pharmaceutical composition of Compound 1 of the present invention, and they may include but are not limited to one or more of the following types of anti-tumor agents: alkylating agents (e.g. carboplatin, oxaliplatin, cisplatin, cyclophosphamide, nitrosoureas, mechlorethamine, melphalan), antimetabolites (e.g. gemcitabine), and antifolates (e.g. 5-fluorouracil and tegafur, raltitrexed, methotrexate, cytarabine, hydroxyurea), topoisomerase inhibitors (e.g. etorposide, topotecan, camptothecin), anti-mitotic agents (e.g. vincristine, vinblastine, vinorelbine, paclitaxel, taxotere), anti-tumor antibiotics (e.g. doxorubicin, bleomycin, doxorubicin, daunorubicin, mitomycin C, actinomycin), anti-estrogens (e.g. tamoxifen, fulvestrant, toremifene, raloxifene, droloxifene), antiandrogens (e.g. bicalutamide, flutamide, nilutamide), LHRH antagonists or LHRH agonists (e.g. goserelin, leuprolide, and buserelin), aromatase inhibitors (such as anastrozole, letrozole), CYP17 lyase inhibitors (such as abiraterone), anti-erbB2 antibody trastuzumab [Herceptin], anti-EGFR antibody cetuximab [Erbitux]; tyrosine kinase, serine/threonine kinase inhibitors (e.g., imatinib and nilotinib, sorafenib, trametinib, crizotinib); cyclin-dependent kinase inhibitors (such as CDK4 inhibitor palbociclib), anti-human vascular endothelial cell growth factor antibody bevacizumab (Avastin) and VEGF receptor tyrosine kinase inhibitor (apatinib), immuno-oncology therapy, such as anti PD-1 antibody (pembrolizumab, nivolumab), anti-PD-L1 antibody, anti-LAG-3 antibody, anti-CTLA-4 antibody, anti-4-1BB antibody, anti-GITR antibody, anti-ICOS antibody, interleukin-2.

### DESCRIPTION OF THE DRAWINGS

FIG. 1 is the XRPD pattern of Compound 1;

FIG. 2 is the DSC and TGA graphs of Compound 1;

FIG. 3 is the NMR spectrum of Compound 1;

FIG. 4 is the XRPD pattern of the crystalline form III of hydrochloride of Compound 1;

FIG. 5 is the DSC and TGA graphs of the crystalline form III of hydrochloride of Compound 1;

FIG. 6 is the 1H NMR spectrum of the crystalline form III of hydrochloride of Compound 1;

FIG. 7 is the DVS plot of the crystalline form III of hydrochloride of Compound 1;

FIG. 8 is the XPRD overlapping pattern of the crystalline form III of hydrochloride of Compound 1 before and after the DVS test;

FIG. 9 is the XRPD pattern of the crystalline form I of phosphate of Compound 1;

FIG. 10 is the DSC and TGA graphs of the crystalline form I of phosphate of Compound 1;

FIG. 11 is the 1H NMR spectrum of the crystalline form I of phosphate of Compound 1;

FIG. 12 is the DVS plot of the crystalline form I of phosphate of Compound 1;

FIG. 13 is the XPRD overlapping pattern of the crystalline form I of phosphate of Compound 1 before and after DVS test;

FIG. 14 is the XRPD pattern of the crystalline form I of p-toluenesulfonate of Compound 1;

FIG. 15 is the DSC and TGA graphs of the crystalline form I of p-toluenesulfonate of Compound 1;

FIG. 16 is the 1H NMR spectrum of the crystalline form I of p-toluenesulfonate of Compound 1;

FIG. 17 is the DVS plot of the crystalline form I of p-toluenesulfonate of Compound 1;

FIG. 18 is the XRPD overlapping pattern of the crystalline form I of p-toluenesulfonate of Compound 1 before and after DVS test;

FIG. 19 is the XRPD pattern of the crystalline form I of benzenesulfonate of Compound 1;

FIG. 20 is the DSC and TGA graphs of the crystalline form I of benzenesulfonate of Compound 1;

FIG. 21 is the 1H NMR spectrum of the crystalline form I of benzenesulfonate of Compound 1;

FIG. 22 is the DVS plot of the crystalline form I of benzenesulfonate of Compound 1;

FIG. 23 is the XPRD overlapping pattern of the crystalline form I of benzenesulfonate of Compound 1 before and after DVS test;

FIG. 24 is the XRPD pattern of the crystalline form I of succinate of Compound 1;

FIG. 25 is the DSC and TGA graphs of the crystalline form I of succinate of Compound 1;

FIG. 26 is the 1H NMR spectrum of the crystalline form I of succinate of Compound 1;

FIG. 27 is the DVS plot of the crystalline form I of succinate of Compound 1;

FIG. 28 is the XPRD overlapping pattern of the crystalline form I of succinate of Compound 1 before and after DVS test;

FIG. 29 is the XRPD pattern of the crystalline form II of succinate of Compound 1;

FIG. 30 is the DSC and TGA graphs of the crystalline form II of succinate of Compound 1;

FIG. 31 is the 1H NMR spectrum of the crystalline form II of succinate of Compound 1;

FIG. 32 is the XRPD pattern of the crystalline form I of sulfate of Compound 1;

FIG. 33 is the DSC and TGA graphs of the crystalline form I of sulfate of Compound 1;

FIG. 34 is the 1H NMR spectrum of the crystalline form I of sulfate of Compound 1;

FIG. 35 is the DVS plot of the crystalline form I of sulfate of Compound 1;

FIG. 36 is the XPRD overlapping pattern of the crystalline form I of sulfate of Compound 1 before and after DVS test;

FIG. 37 is the XRPD pattern of the crystalline form I of monohydrobromide of Compound 1;

FIG. 38 is the DSC and TGA graphs of the crystalline form I of monohydrobromide of Compound 1;

FIG. 39 is the 1H NMR spectrum of the crystalline form I of monohydrobromide of Compound 1;

FIG. 40 is the XRPD pattern of the crystalline form I of dihydrobromide of Compound 1;

FIG. 41 is the DSC and TGA graphs of the crystalline form I of dihydrobromide of Compound 1;

FIG. 42 is the 1H NMR spectrum of the crystalline form I of dihydrobromide of Compound 1.

### ADVANTAGEOUS EFFECTS

The inventor has discovered by accident that for p-toluenesulfonate, benzenesulfonate, succinate, hydrochloride, phosphate, and sulfate of Compound 1, a mono-salt can be formed in high yields at a molar ratio of the compound to the corresponding acid of slightly less than 1:1, such as 1:1.1 (acid excess), so the process scale-up is simplified and the efficiency is improved.

In addition, as detailed herein, compared with Compound 1, some salt forms of Compound 1, such as hydrochloride, phosphate, p-toluenesulfonate, benzenesulfonate, succinate, sulfate, hydrobromide (including monohydrobromide or dihydrobromide), have more or less improved water solubility, and some polymorphs of these salt forms (especially p-toluenesulfonate crystalline form I, benzenesulfonate crystalline form I, phosphate crystalline form I, etc.) have properties such as high stability, low moisture absorption, which is beneficial to the production and preparation of Compound 1, and is of great significance to its final marketization.

### DETAILED EMBODIMENTS

The present invention is further illustrated by the following examples. The following examples are just used to more specifically illustrate the preferred embodiments of the present invention, and are not used to limit the technical solutions of the present invention.

In the following examples,

[symbol]

The instrument used in ¹H-NMR analysis was a Bruker Advance 300 equipped with a B-ACS 120 automatic sampling system.

The solid samples were analyzed with a powder X-ray diffraction analyzer (Bruker D8 advance). The instrument is equipped with a LynxEye detector. The 20 scan angle range is 3° to 40°, and the step size is 0.02°. When measuring the sample, the light tube voltage and light tube current were 40 KV and 40 mA, respectively.

The instrument used in thermogravimetric analysis (TGA) was Discovery TGA 55 (TA Instruments, US). The sample was placed in a balanced open aluminum sample pan, and the sample was automatically weighed in the TGA furnace. The sample was heated to the final temperature at a rate of 10° C./min

The instrument used in differential scanning calorimetry (DSC) was TA Instruments Q200 or Discovery DSC 250. After the sample was accurately weighed, it was placed in a DSC sample pan with a pierced lid, and the mass of the sample was accurately recorded. The sample was heated to the final temperature at a heating rate of 10° C./min.

The instrument used in dynamic vapor absorption and desorption analysis (DVS) was DVS Intrinsic (SMS, UK). The sample was placed in the sample basket of the instrument for automatic weighing, then heated to 40° C., and dried under a nitrogen stream to a dm/dt of less than 0.002%. The measurement was started after the temperature was dropped to 25° C., The instrument parameters were as follows.

Time per step: 60 min
Sample temperature: 25° C.
Cycle: entire cycle

### Adsorption: 0, 10, 20, 30, 40, 50, 60, 70, 80, 90

### Desorption: 80, 70, 60, 50, 40, 30, 20, 10, 0

Data storage rate: 5 s
Total flow rate: 200 sccm
Total flow rate after the test: 200 sccm

### Characterization of Compound 1

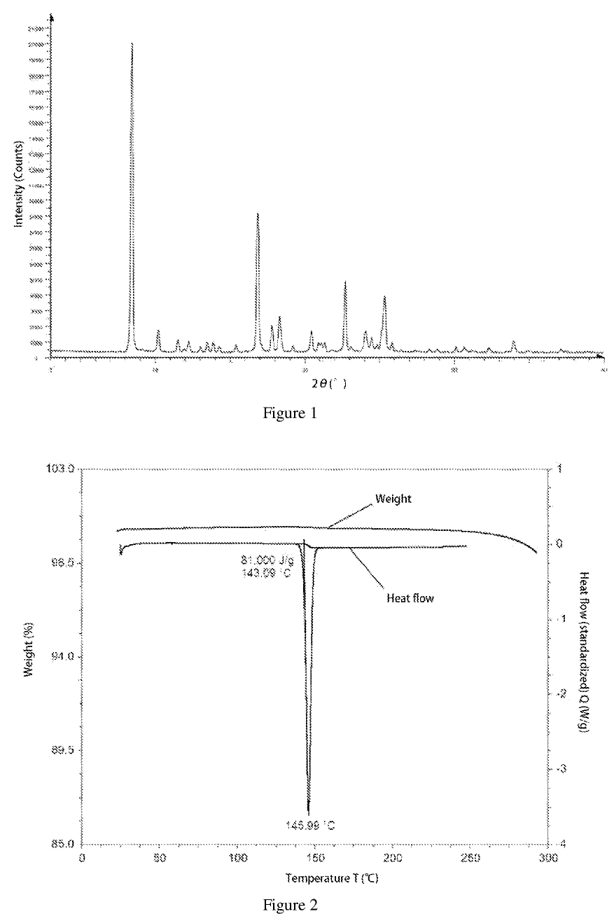

The initial drug 1 is a crystal with good crystallinity (FIG. 1), and its melting point is 146° C. as shown in DSC (FIG. 2). The sample has no residual solvent and almost no weight loss at temperatures lower than 200° C. as shown in ¹H-NMR and TGA (FIG. 3). The results show that the sample is an anhydrous crystal, named as crystalline form I.

### Preparation of Various Salt Forms

### Example 1. Crystalline Form III of Hydrochloride

1 (31.21 mg, 1.0 eq) was dissolved in a mixed solvent of acetonitrile and dichloromethane (48 v, 3/1), and hydrochloric acid (1.1 eq) was added under stirring at 50° C. After the reaction solution was cooled to room temperature, the solution was stirred for 30 minutes. Then the resulting clear solution was concentrated to about 32 v with N₂ stream, and a solid precipitated out immediately. The resulting suspension was stirred overnight at room temperature, and a solid was collected by filtration, and dried under vacuum at 50° C. for about 4 hours to obtain a crystalline form III of hydrochloride, which sample, an off-white solid, was characterized by XRPD, DSC, TGA, DVS and ¹H-NMR, respectively.

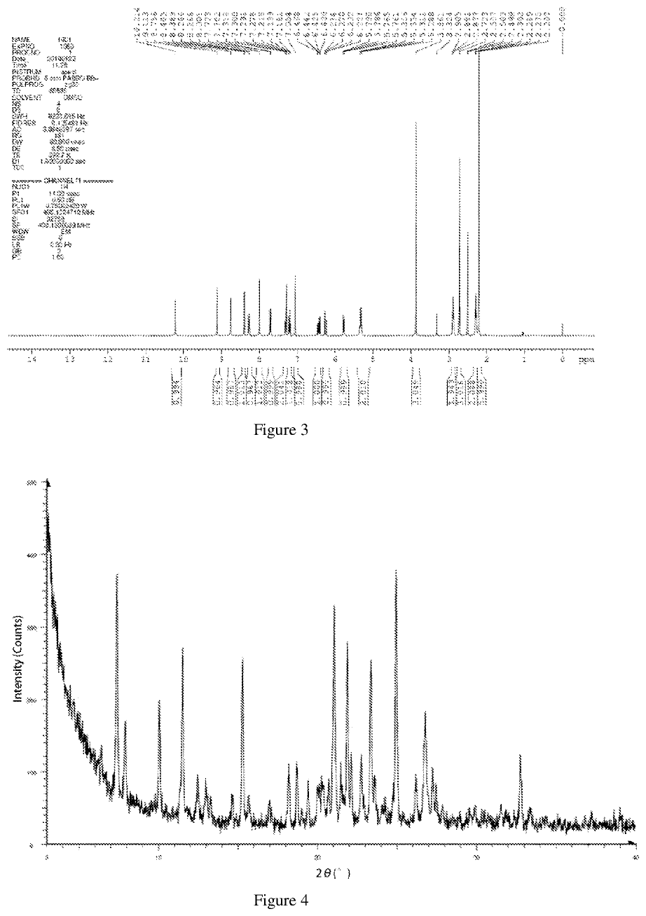

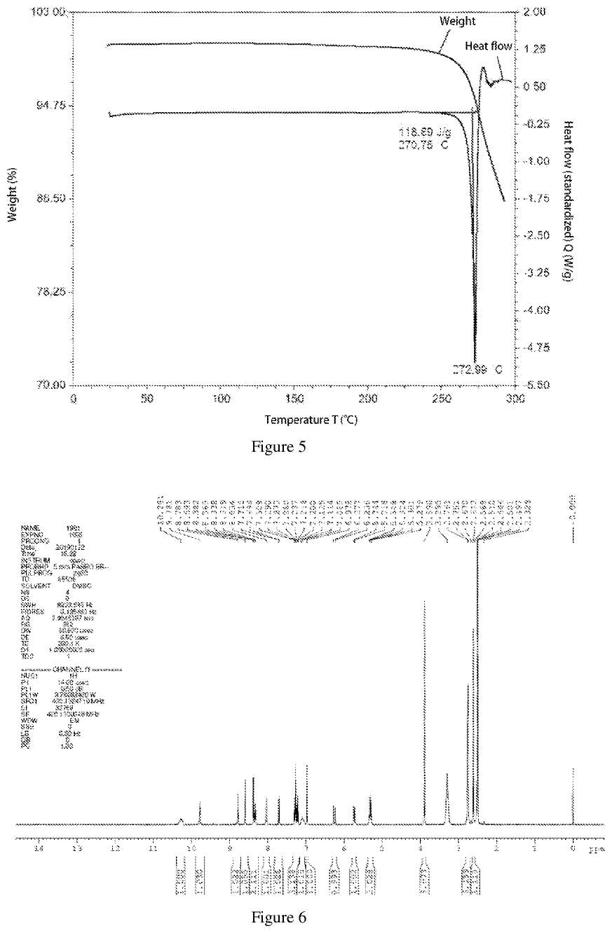

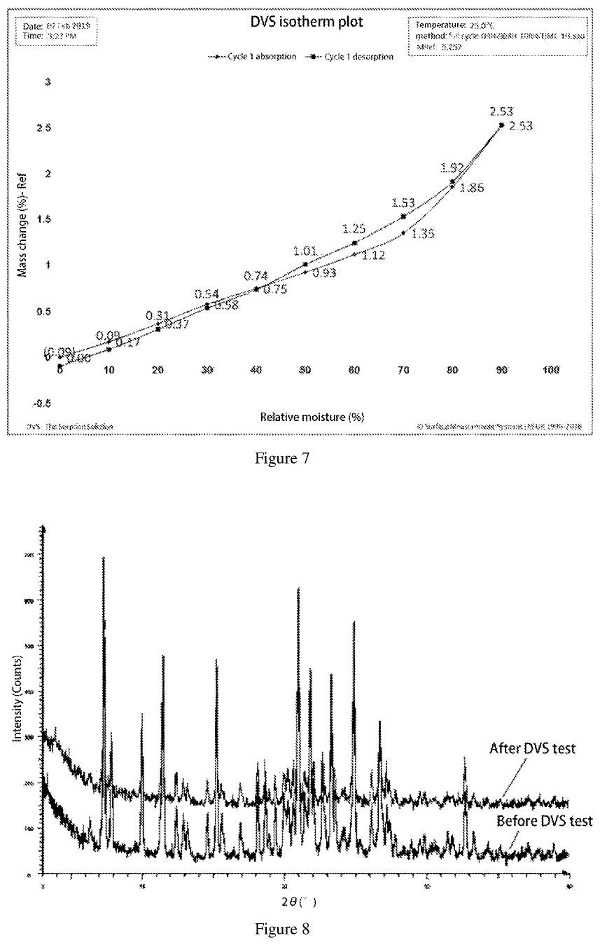

The crystalline form III of hydrochloride is a crystal with a high melting point (273° C., FIG. 5) (Table 1 and FIG. 4). The sample is slightly hygroscopic, with a weight gain of about 1.86% under 80% relative humidity (FIG. 7). The sample has no residual solvent and no significant weight loss at temperature lower than 200° C. as shown in ¹H-NMR and TGA (FIG. 5 and FIG. 6), indicating that the sample is an anhydrous crystal. The crystalline form of the sample does not change after the DVS test (FIG. 8).

TABLE 1
List of XRPD diffraction peaks of the crystalline form
III of hydrochloride

| Angle | Intensity | Angle | Intensity | Angle | Intensity |
|---|---|---|---|---|---|
| 2θ/° | % | 2θ/° | % | 2θ/° | % |
| 6.385 | 35.9 | 18.713 | 27.2 | 24.944 | 100 |
| 7.353 | 98.4 | 19.4 | 23.2 | 26.206 | 25.1 |
| 7.872 | 44.9 | 20.042 | 21.1 | 26.789 | 48 |
| 10.033 | 52.2 | 20.313 | 22.4 | 27.255 | 28 |
| 11.483 | 71.5 | 20.694 | 23.7 | 27.481 | 21.1 |
| 12.445 | 25.1 | 21.037 | 87.3 | 27.875 | 14 |
| 12.977 | 20.8 | 21.485 | 21.4 | 28.937 | 11.9 |
| 13.255 | 17.2 | 21.867 | 73.6 | 29.921 | 13.7 |
| 14.632 | 17.9 | 22.134 | 33 | 31.559 | 14.5 |
| 15.266 | 67.5 | 22.745 | 32.7 | 32.794 | 32.2 |
| 15.657 | 17.4 | 23.353 | 67 | 33.388 | 13.2 |
| 16.947 | 15.8 | 23.621 | 24.8 | 37.271 | 11.3 |
| 18.181 | 29 | 24.101 | 14.2 | 39.086 | 9.8 |

### Example 2. Crystalline Form I of Phosphate

1 (30.20 mg, 1.0 eq) was dissolved in acetone (26 v), and phosphoric acid (1.1 eq) was added under stirring at room temperature, and a viscous substance immediately precipitated out. After stirring for 2 hours, a solid precipitated out. After the suspension was stirred at room temperature for 3 hours, a solid was collected by filtration and dried in vacuum at 50° C. overnight to obtain crystalline form I of phosphate, which sample, an off-white solid, was characterized by XRPD, DSC, TGA, DVS and ¹H-NMR, respectively.

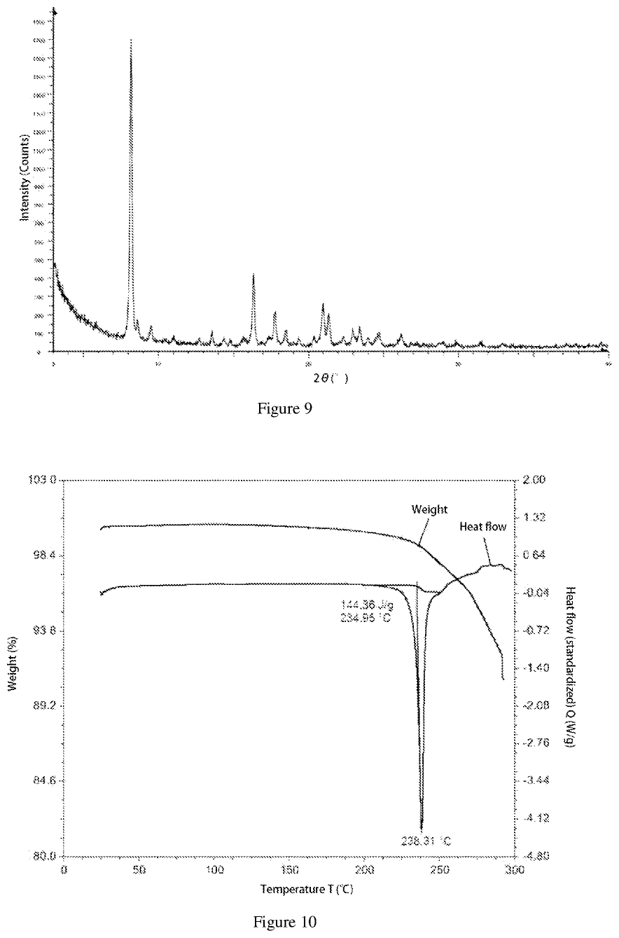

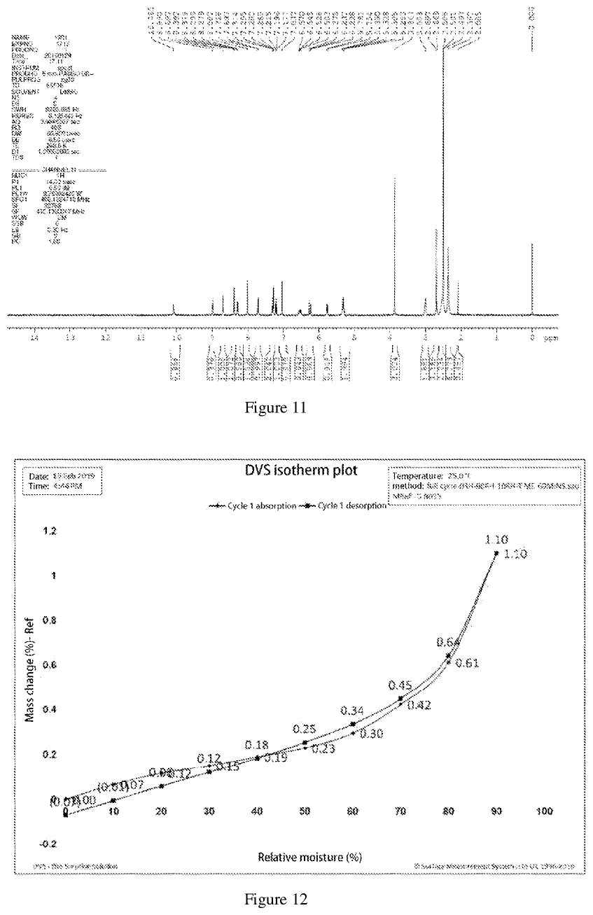

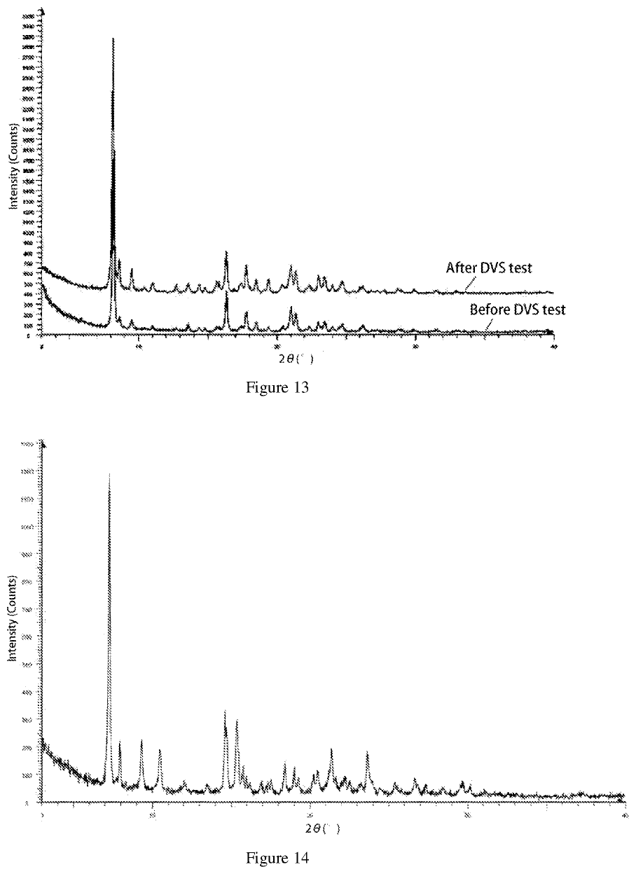

The crystalline form I of phosphate is a crystal with high crystallinity (Table 2 and FIG. 9) and high melting point (238° C., FIG. 10). The sample is slightly hygroscopic, with a weight gain of about 0.61% under 80% relative humidity (FIG. 12). The sample has 0.7% residual solvent, and no significant weight loss at temperatures lower than 150° C. as shown in ¹H-NMR and TGA (FIG. 10 and FIG. 11), indicating that the sample is an anhydrous crystal. The crystalline form of the sample does not change after the DVS test (FIG. 13).

TABLE 2
List of XRPD diffraction peaks of crystalline form I of phosphate

| Angle | Intensity | Angle | Intensity | Angle | Intensity |
|---|---|---|---|---|---|
| 2θ/° | % | 2θ/° | % | 2θ/° | % |
| 8.144 | 100 | 17.395 | 4.6 | 24.015 | 4.3 |
| 8.573 | 10 | 17.752 | 12.6 | 24.715 | 5 |
| 9.48 | 8.1 | 18.48 | 6.6 | 26.218 | 5.4 |
| 10.988 | 5.1 | 19.362 | 4.3 | 26.91 | 2.1 |
| 12.698 | 4 | 20.389 | 4.8 | 29.013 | 2.9 |
| 13.554 | 6.5 | 20.994 | 15.7 | 29.882 | 3.3 |
| 14.334 | 4 | 21.366 | 11.9 | 31.536 | 2.3 |
| 14.767 | 3.6 | 22.361 | 4.7 | 32.976 | 2.4 |
| 15.671 | 4.8 | 22.992 | 7.3 | 37.285 | 2.2 |
| 16.316 | 24.5 | 23.451 | 7.9 | 39.543 | 2.3 |

### Example 3. Crystalline Form I of p-toluenesulfonate

1 (31.60 mg, 1.0 eq) was dissolved in acetone (25 v), and p-toluenesulfonic acid (1.1 eq) was added under stirring at room temperature. After about 2 minutes, a solid precipitated out. The suspension was stirred at room temperature for about 6 hours, and a solid was collected by filtration and dried overnight at 50° C. under vacuum to obtain a crystalline form I of p-toluenesulfonate, which sample, an off-white solid, was characterized by XRPD, DSC, TGA, DVS and ¹H-NMR, respectively.

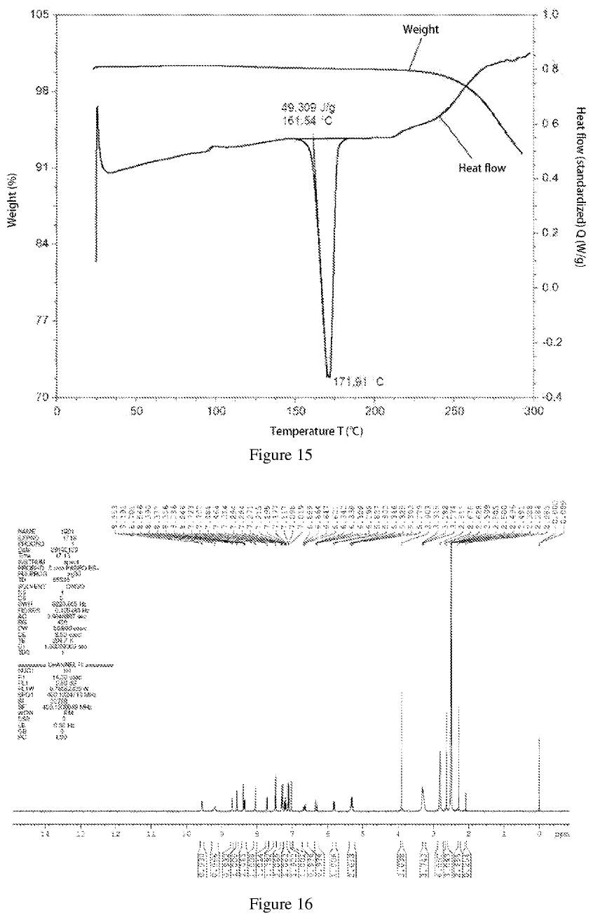

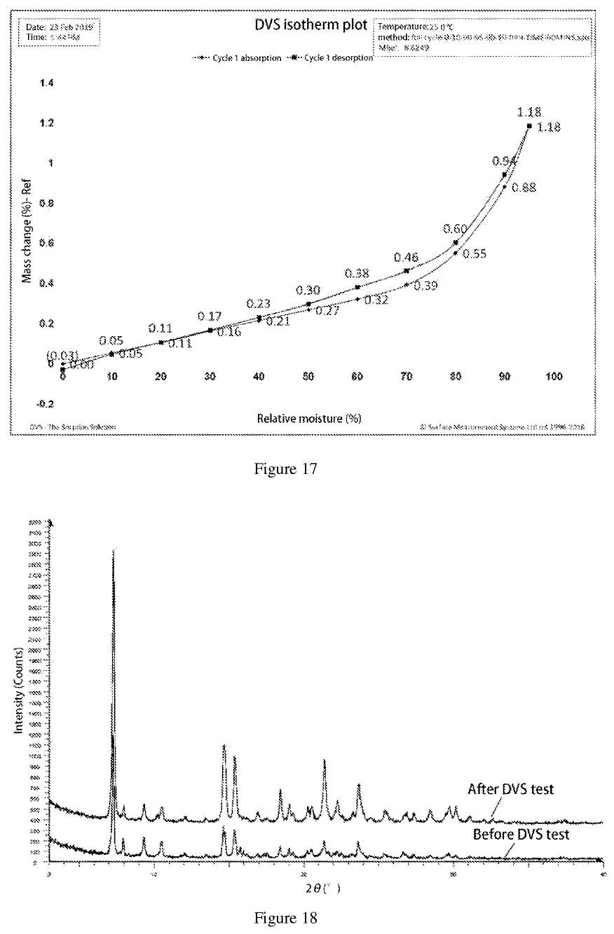

The crystalline form I of p-toluenesulfonate is a crystal with a melting point of 172° C. (FIG. 15) (Table 3 and FIG. 14). The sample is slightly hygroscopic, with a weight gain of about 0.55% under 80% relative humidity (FIG. 17). The sample has no significant weight loss at temperatures lower than 200° C. as shown in TGA (FIG. 15); the sample has about 0.3% residual solvent, and the ratio of free base to p-toluenesulfonic acid is 1:1 as shown in ¹H-NMR (FIG. 16). The sample may be an anhydrous crystal. The crystalline form of the sample does not change after the DVS test (FIG. 18).

TABLE 3
List of XRPD diffraction peaks of crystalline form I
of p-toluenesulfonate

| Angle | Intensity | Angle | Intensity | Angle | Intensity |
|---|---|---|---|---|---|
| 2θ/° | % | 2θ/° | % | 2θ/° | % |
| 7.221 | 100 | 17.536 | 6.7 | 23.679 | 14.2 |
| 7.904 | 18.5 | 18.385 | 11.8 | 24.457 | 4.5 |
| 9.293 | 18.7 | 19.004 | 10.4 | 25.408 | 5.8 |
| 10.459 | 15.6 | 19.25 | 7.3 | 26.66 | 7.1 |
| 12.015 | 6.3 | 20.231 | 8.4 | 27.37 | 5.1 |
| 13.478 | 4.5 | 20.498 | 9.5 | 28.449 | 4.3 |
| 14.638 | 23.3 | 21.368 | 16.3 | 29.728 | 6.1 |
| 15.36 | 24.7 | 22.224 | 7.6 | 30.176 | 4.1 |
| 15.708 | 9.2 | 22.529 | 6.5 | 31.107 | 3.7 |
| 16.892 | 5.2 | 23.184 | 4.4 | | |

### Example 4. Crystalline Form I of Benzenesulfonate

1 (19.51 mg, 1.0 eq) was dissolved in acetone (40 v), and benzenesulfonic acid (1.0 eq) was added under stirring at room temperature. The reaction solution was still clear after stirring for 3 hours. It was blown dry with N₂ stream. The resulting viscous substance was suspended in acetonitrile (50 v) at room temperature and slurried overnight. A solid was collected by filtration and dried under vacuum at 50° C. for about 4 hours to obtain a crystalline form I of benzenesulfonate, which sample, a white solid, was characterized by XRPD, DSC, TGA, DVS and ¹H-NMR, respectively.

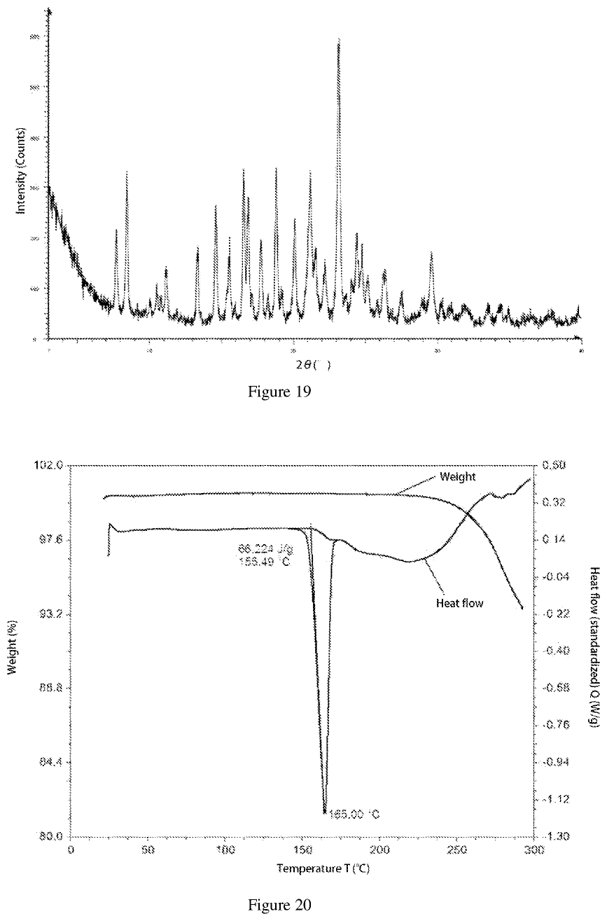

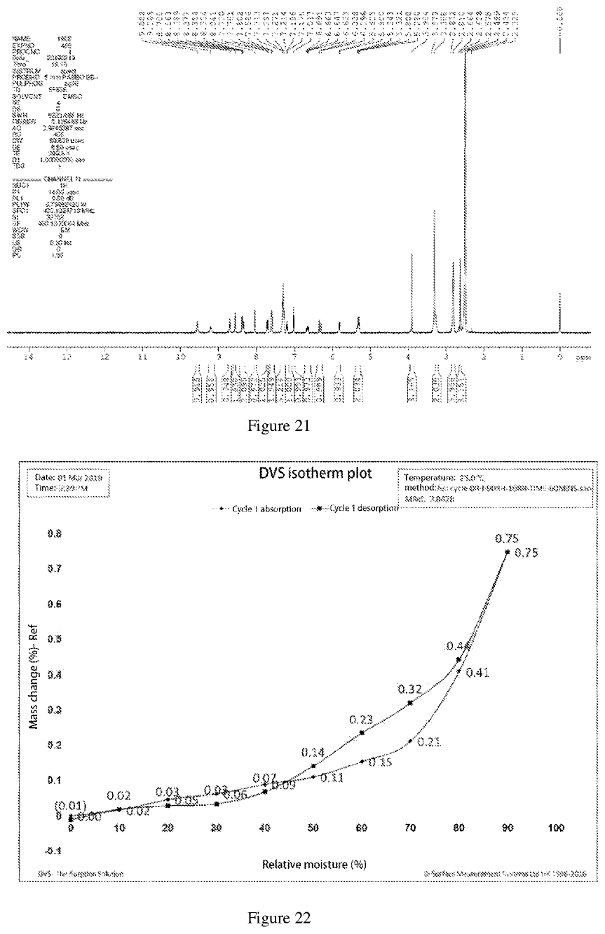

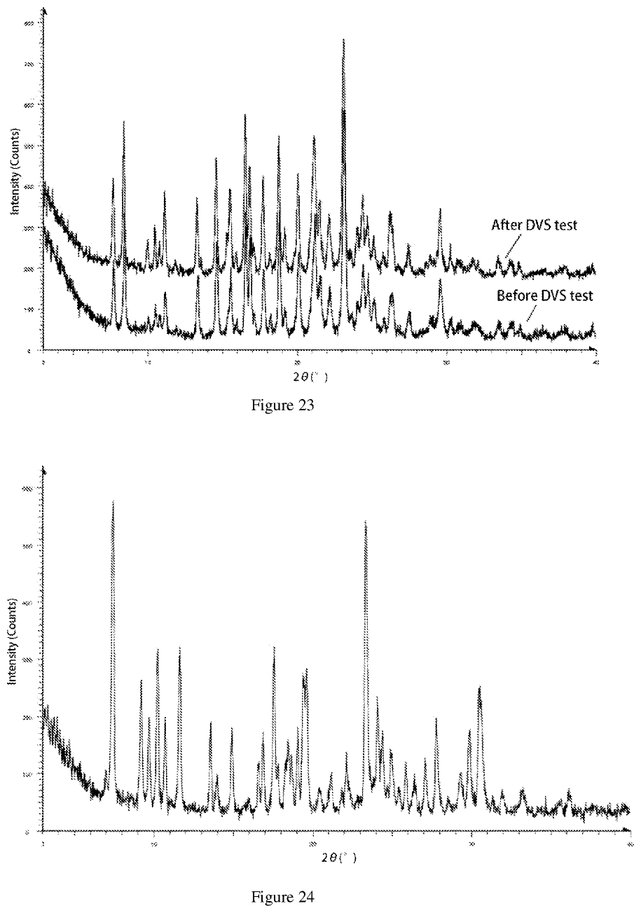

The crystalline form I of benzenesulfonate is a crystal with a melting point of 165° C. (FIG. 20) (Table 4 and FIG. 19). The sample is slightly hygroscopic, with a weight gain of about 0.41% under 80% relative humidity (FIG. 22). The sample has no significant weight loss at temperatures lower than 180° C. as shown in TGA (FIG. 20); the sample has no residual solvent, and the ratio of free base to benzenesulfonic acid was 1:1 as shown in ¹H-NMR (FIG. 21). The sample is an anhydrous crystal, and the crystalline form of the sample does not change after the DVS test (FIG. 23).

TABLE 4
List of XRPD diffraction peaks of crystalline form I of
benzenesulfonate

| Angle | Intensity | Angle | Intensity | Angle | Intensity |
|---|---|---|---|---|---|
| 2θ/° | % | 2θ/° | % | 2θ/° | % |
| 7.675 | 36.4 | 17.122 | 14.6 | 24.769 | 31.4 |
| 8.411 | 55.9 | 17.728 | 32.9 | 25.162 | 21 |
| 10.009 | 13.3 | 18.196 | 13.1 | 25.846 | 12.8 |
| 10.494 | 18.3 | 18.782 | 56.9 | 26.396 | 23 |
| 10.766 | 12.8 | 19.181 | 14.4 | 27.523 | 15.3 |
| 11.143 | 23.7 | 20.084 | 39.8 | 29.625 | 28 |
| 13.3 | 28.4 | 21.177 | 55.7 | 30.277 | 12.2 |
| 14.595 | 44.3 | 21.532 | 29.5 | 33.549 | 10.2 |
| 15.523 | 33.7 | 22.191 | 26.5 | 34.355 | 11.4 |
| 15.89 | 10.1 | 23.163 | 100 | 34.441 | 11.4 |
| 16.534 | 56.4 | 24.082 | 19.6 | 39.824 | 10.9 |
| 16.845 | 47 | 24.415 | 34.7 | | |

### Example 5. Crystalline Form I of Succinate

1 (31.3 mg, 1.0 eq) was dissolved in acetone (26 v), and succinic acid (1.1 eq, 0.6 M in methanol) was added under stirring at room temperature. The reaction solution was still clear after stirring for 2 hours. It was blown dry with N₂ stream. The resulting viscous substance was suspended and slurried in acetonitrile (16 v) at room temperature for 2 hours. A solid was collected by filtration and dried under vacuum at 50° C. overnight to obtain a crystalline form I of succinate, which sample, an off-white solid, was characterized by XRPD, DSC, TGA, DVS and ¹H-NMR, respectively.

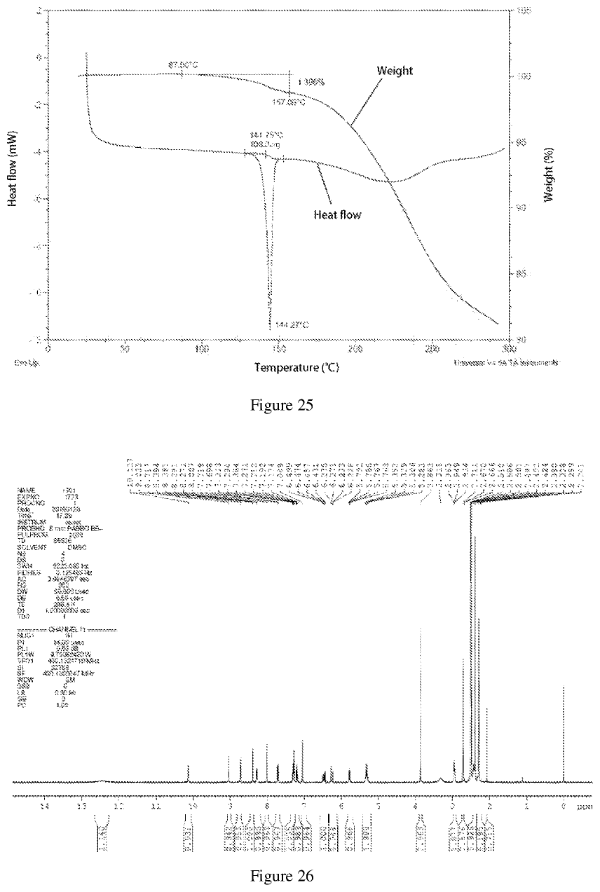

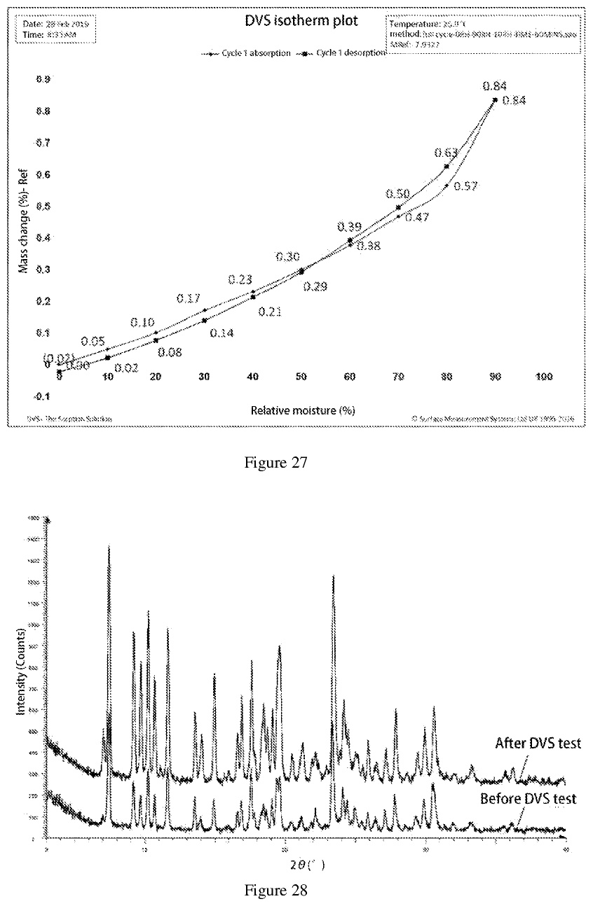

The crystalline form I of succinate is a crystal with high crystallinity (Table 5 and FIG. 24), and with a melting point of 144° C. (FIG. 25). The sample is slightly hygroscopic, with a weight gain of about 0.57% under 80% relative humidity (FIG. 27).The sample loses about 1.4% in weight between 87 and 157° C. as shown in TGA (FIG. 25), and the sample has about 1% residual solvent, and the ratio of free base to succinic acid is 1:1 as shown in ¹H-NMR (FIG. 26). The sample is an anhydrous crystal, and the crystalline form of the sample does not change after DVS test (FIG. 28).

TABLE 5
List of XRPD diffraction peaks of crystalline form I of succinate

| Angle | Intensity | Angle | Intensity | Angle | Intensity |
|---|---|---|---|---|---|
| 2θ/° | % | 2θ/° | % | 2θ/° | % |
| 6.946 | 18.5 | 17.811 | 20.6 | 25.463 | 13.2 |
| 7.376 | 100 | 18.449 | 27.6 | 25.892 | 20.7 |
| 9.175 | 46.2 | 18.642 | 23.2 | 26.463 | 17.2 |
| 9.674 | 34.8 | 19.051 | 31.8 | 27.119 | 22.5 |
| 10.209 | 56.1 | 19.42 | 47.3 | 27.829 | 31.6 |
| 10.672 | 35 | 19.595 | 49.9 | 28.567 | 10 |
| 11.594 | 56.8 | 20.418 | 13.2 | 29.326 | 17.8 |
| 13.549 | 33.4 | 21.142 | 16.9 | 29.9 | 31.1 |
| 13.952 | 17.4 | 21.864 | 13 | 30.547 | 44.8 |
| 14.89 | 31.5 | 22.144 | 24.1 | 31.357 | 10.7 |
| 15.942 | 10.2 | 23.376 | 93.8 | 31.958 | 12.7 |
| 16.57 | 20.2 | 24.111 | 41.3 | 33.223 | 12.3 |
| 16.859 | 30.4 | 24.402 | 30.8 | 35.668 | 9.7 |
| 17.554 | 56.8 | 24.975 | 23.6 | 36.201 | 12.7 |

### Example 6. Crystalline Form II of Succinate

1 (30.2 mg, 1.0 eq) was dissolved in ethyl acetate (33 v), and succinic acid (1.1 eq, 0.6 M in methanol) was added under stirring at 35° C. The reaction solution was still clear after stirring for 2 hours. It was blown dry with N₂ stream. The resulting viscous substance was suspended and slurried in 2-butanone (16 v) at room temperature overnight. A solid was collected by filtration and dried under vacuum at 50° C. for about 4 hours to obtain a crystalline form II of succinate, which sample, a white solid, was characterized by XRPD, DSC, TGA and ¹H-NMR, respectively.

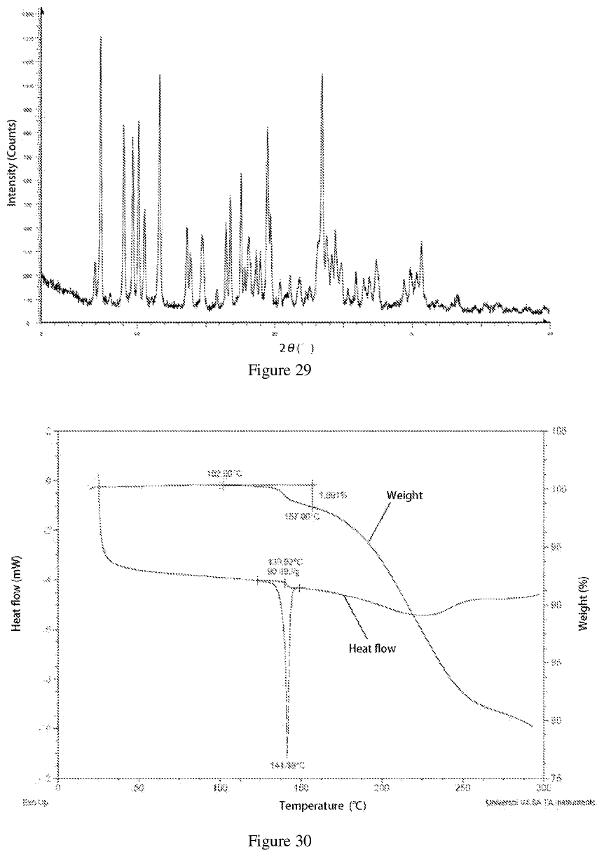

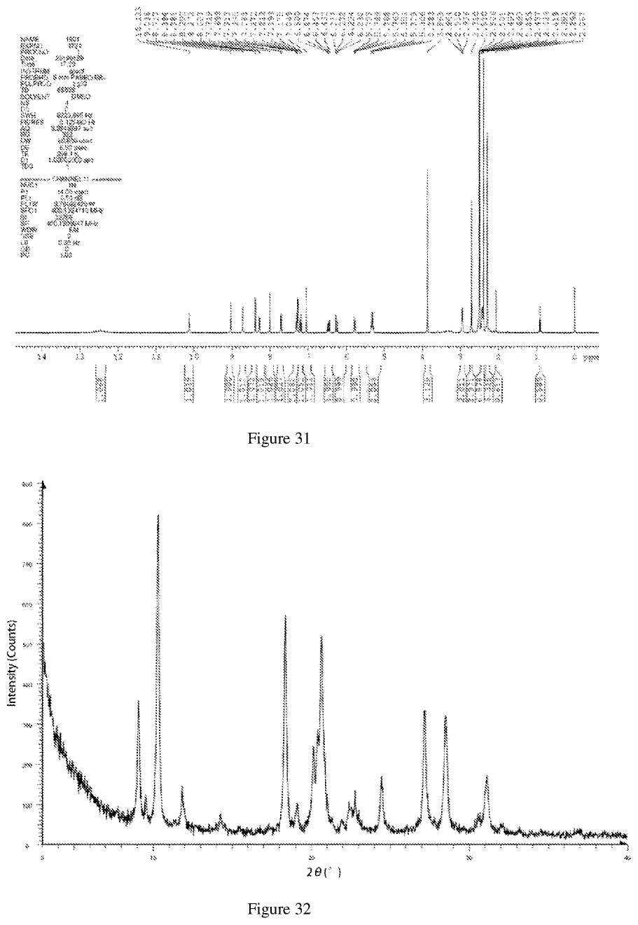

The crystalline form II of succinate is a crystalline with high crystallinity (Table 6 and FIG. 29), with a melting point of 141° C. (FIG. 30). The sample has a weight loss of about 1.9% between 102 and 157° C. as shown in TGA (FIG. 30); the sample has about 2% residual 2-butanone, and the ratio of free base to succinic acid is 1:1 as shown in ¹H-NMR (FIG. 31). The sample is an anhydrous crystal.

TABLE 6
List of XRPD diffraction peaks of crystalline form II of succinate

| Angle | Intensity | Angle | Intensity | Angle | Intensity |
|---|---|---|---|---|---|
| 2θ/° | % | 2θ/° | % | 2θ/° | % |
| 6.89 | 21.4 | 18.644 | 25.4 | 26.482 | 15.5 |
| 7.321 | 100 | 18.945 | 24.9 | 26.897 | 16 |
| 8.014 | 10.2 | 19.474 | 68.2 | 27.402 | 21.6 |
| 9.022 | 69 | 19.702 | 37.8 | 28.108 | 7 |
| 9.652 | 65.1 | 20.376 | 13.9 | 29.431 | 14.8 |
| 10.087 | 70.4 | 21.106 | 16.4 | 29.892 | 19 |
| 10.51 | 39.4 | 21.8 | 15.7 | 30.33 | 17.4 |
| 11.63 | 86.7 | 22.293 | 10.6 | 30.688 | 28.3 |
| 13.604 | 33.5 | 22.561 | 12.6 | 31.826 | 7.2 |
| 13.881 | 24.4 | 23.148 | 28 | 33.307 | 9.9 |
| 14.734 | 30.7 | 23.454 | 87.2 | 34.561 | 6 |
| 15.781 | 11.8 | 23.786 | 30.7 | 35.276 | 7 |
| 16.446 | 35 | 24.171 | 23.5 | 36.167 | 6.7 |
| 16.774 | 44.3 | 24.428 | 32.2 | 36.427 | 6.1 |
| 17.534 | 52.1 | 24.839 | 20.8 | 39.608 | 5.8 |
| 17.821 | 19.3 | 25.349 | 11.8 | | |
| 18.131 | 30.1 | 25.942 | 17.4 | | |

### Example 7. Crystalline Form I of Sulfate

1 (29.80 mg, 1.0 eq) was dissolved in ethyl acetate (33 v), and sulfuric acid (1.0 eq, 0.1 M in methanol) was added under stirring at 35° C. A solid precipitated out immediately. After the suspension was cooled to room temperature, and stirred overnight, a solid was collected by filtration and dried in vacuum at 50° C. for about 4 hours to obtain a crystalline form I of sulfate, which sample, a light yellow solid, was characterized by XRPD, DSC, TGA, DVS and ¹H-NMR, respectively.

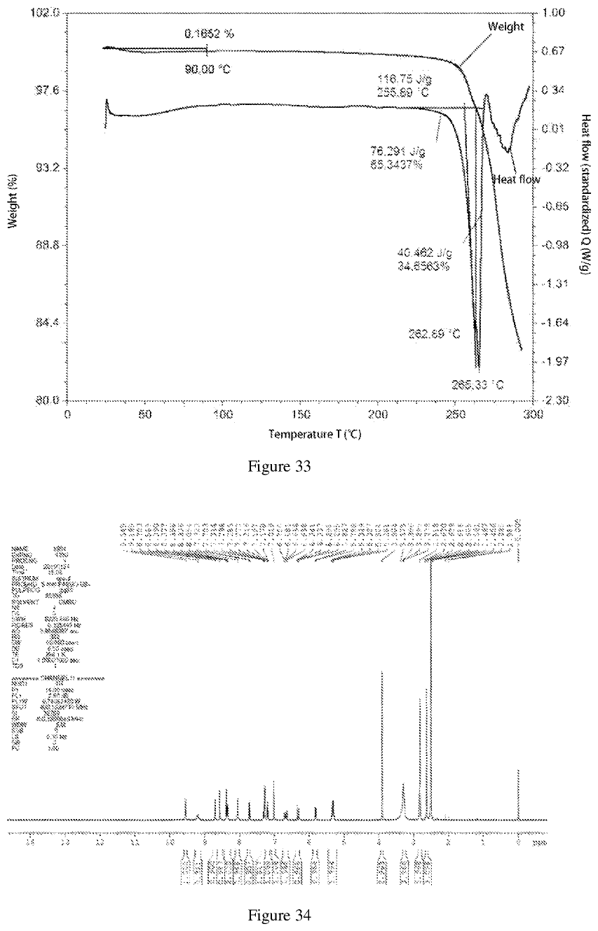

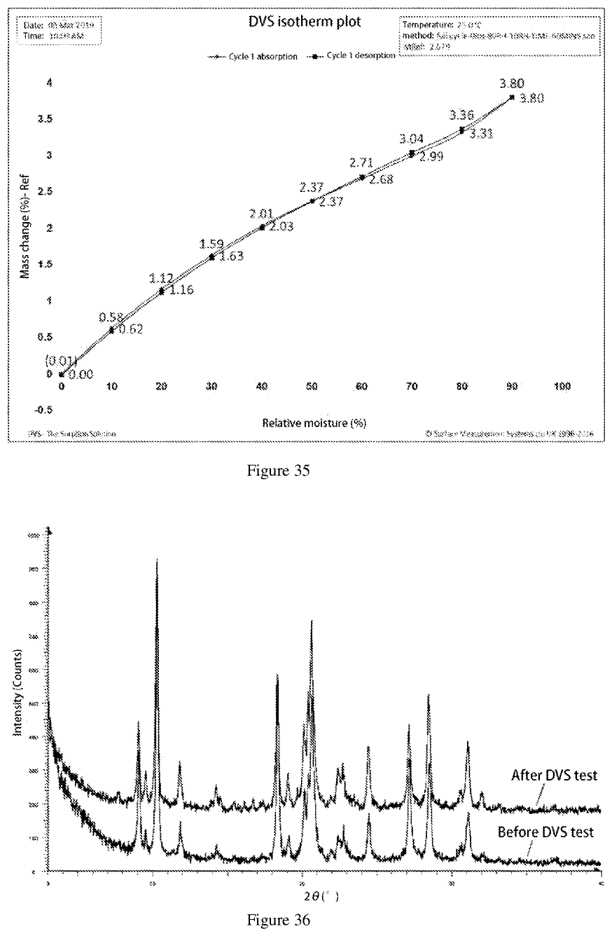

The crystalline form I of sulfate is a crystal with good crystallinity (Table 7 and FIG. 32). There are two overlapping endothermic peaks at 263° C. and 265° C. (FIG. 33), which may be results of crystalline transformations of the sample during the heating process. The sample is hygroscopic and gains about 3.31% in weight under 80% relative humidity (FIG. 35). The sample has a weight loss of 0.2% between room temperature and 90° C. as shown in TGA; the sample had about 0.3% residual ethyl acetate as shown in ¹H-NMR (FIG. 33 and FIG. 34). The sample may be an anhydrous crystal. The crystalline form of the sample does not change after the DVS test (FIG. 36).

TABLE 7
List of XRPD diffraction peaks of crystalline form I of sulfate

| Angle | Intensity | Angle | Intensity | Angle | Intensity |
|---|---|---|---|---|---|
| 2θ/° | % | 2θ/° | % | 2θ/° | % |
| 9.039 | 43.4 | 20.141 | 29.4 | 27.196 | 40.3 |
| 9.49 | 14.7 | 20.411 | 34.9 | 28.534 | 38.8 |
| 10.275 | 100 | 20.635 | 63.3 | 30.647 | 8.4 |
| 11.809 | 17.6 | 21.261 | 8.4 | 31.141 | 20.7 |
| 14.239 | 9.5 | 21.943 | 7.5 | 32.097 | 5.6 |
| 15.432 | 5.1 | 22.45 | 10.3 | 33.216 | 5 |
| 18.342 | 69.1 | 22.792 | 16.3 | | |
| 19.085 | 12.6 | 24.479 | 20.4 | | |

### Example 8. Crystalline Form I of Monohydrobromide

1 (32.0 mg, 1.0 eq) was dissolved in acetone (22 v), hydrobromic acid (1.1 eq) was added under stirring at room temperature. After the reaction solution was stirred for 5 minutes, a solid precipitated out. The suspension was stirred for about 2 hours, and a solid was collected by filtration and dried overnight at 50° C. under vacuum to obtain a crystalline form I of monohydrobromide, which sample, an orange-yellow solid, was characterized by XRPD, DSC, TGA and ¹H-NMR, respectively.

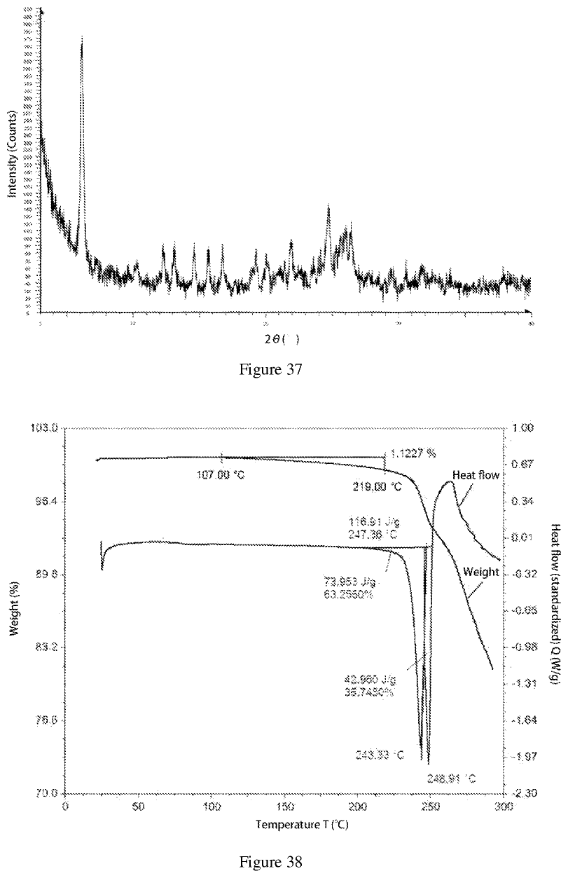

The crystalline form I of monohydrobromide is a crystal with relatively poor crystallinity (Table 8 and FIG. 37). There are two overlapping endothermic peaks at 243° C. and 249° C. (FIG. 38), which may be results of crystalline transformations of the sample during the heating process. The sample has a 1.1% weight loss between 107 and 219° C. as shown in TGA, and the sample has about 1.2% residual acetone as shown in ¹H-NMR (FIG. 38 and FIG. 39). The sample is an anhydrous crystal.

TABLE 8
List of XRPD diffraction peaks of crystalline form I of
monohydrobromide

| Angle | Intensity | Angle | Intensity | Angle | Intensity |
|---|---|---|---|---|---|
| 2θ/° | % | 2θ/° | % | 2θ/° | % |
| 3.67 | 49.7 | 17.703 | 11.8 | 28.981 | 12 |
| 6.104 | 100 | 19.27 | 23 | 29.532 | 15.8 |
| 10.262 | 18.7 | 20.057 | 21.1 | 30.584 | 18.7 |
| 12.251 | 24.6 | 21.916 | 26.2 | 31.816 | 17.4 |
| 13.07 | 20.9 | 23.634 | 16.8 | 31.923 | 15 |
| 14.58 | 24.9 | 24.73 | 39 | 37.951 | 13.6 |
| 15.651 | 23.3 | 26.032 | 28.9 | 39.358 | 13.4 |
| 16.739 | 24.6 | 26.437 | 32.6 | | |

### Example 9. Crystalline Form I of Dihydrobromide

1 (31.96 mg, 1.0 eq) was dissolved in acetone (38 v), hydrobromic acid (2.0 eq) was added under stirring at 50° C. The reaction solution was still clear after stirring for 2 hours. The solvent was removed by rotary evaporation and the resulting viscous substance was suspended in acetonitrile (45 v) at room temperature and slurried overnight. A solid was collected by filtration and dried under vacuum at 50° C. for about 4 hours to obtain a crystalline form I of dihydrobromide, which sample, an orange-yellow solid, was characterized by XRPD, DSC, TGA and ¹H-NMR, respectively.

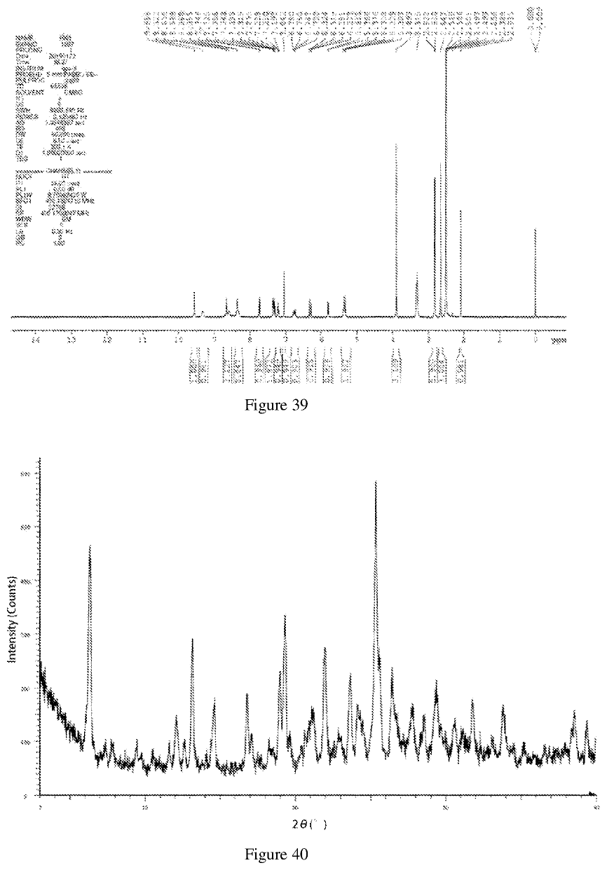

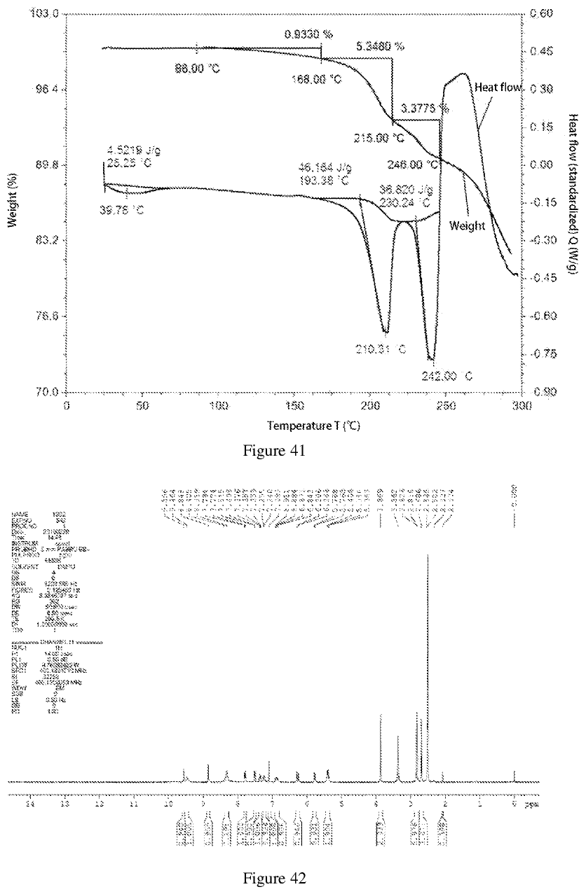

The salt form I of dihydrobromide is a crystal with good crystallinity (Table 9 and FIG. 40). There are two overlapping endothermic peaks at 210° C. and 242° C. (FIG. 41), which may be results of crystalline transformations of the sample during the heating process; in addition, there is a broad endothermic peak at 25-40° C., which may be result of the loss of solvent or water, and this part of solvent or water is easily lost. TGA shows three weight losses (FIG. 41). The first weight loss may be result of solvent loss. The sample has about 0.9% residual acetonitrile as shown in ¹H-NMR (FIG. 42); and the last two weight losses may be caused by decomposition. The sample may be an anhydrous crystal.

TABLE 9
List of XRPD diffraction peaks of crystalline form I of
dihydrobromide

| Angle | Intensity | Angle | Intensity | Angle | Intensity |
|---|---|---|---|---|---|
| 2θ/° | % | 2θ/° | % | 2θ/° | % |
| 6.276 | 79.5 | 18.953 | 39.5 | 28.58 | 25.5 |
| 7.329 | 17.4 | 19.305 | 57.3 | 29.384 | 36.4 |
| 7.771 | 16.1 | 19.605 | 20.3 | 30.618 | 23.4 |
| 9.38 | 14.7 | 20.387 | 15.6 | 31.164 | 21.4 |
| 9.69 | 13.3 | 20.662 | 19 | 31.832 | 29.2 |
| 10.493 | 14.5 | 21.148 | 27.2 | 32.348 | 20 |
| 11.591 | 17.1 | 21.954 | 46.8 | 33.126 | 17.3 |
| 12.071 | 25.3 | 22.87 | 21.4 | 33.849 | 28.4 |
| 12.603 | 17.6 | 23.626 | 37.1 | 34.543 | 16.4 |
| 13.122 | 50.1 | 24.148 | 28.4 | 35.211 | 16.8 |
| 14.575 | 28.5 | 25.341 | 100 | 36.629 | 15.9 |
| 16.777 | 32.1 | 25.61 | 43.6 | 38.6 | 26.8 |
| 17.067 | 19.5 | 26.424 | 40.5 | 39.414 | 23.6 |
| 18.236 | 17.9 | 27.78 | 29.1 | | |

1. A salt of Compound 1, which is a p-toluenesulfonate, benzenesulfonate, succinate, hydrochloride, phosphate, sulfate, or hydrobromide salt:

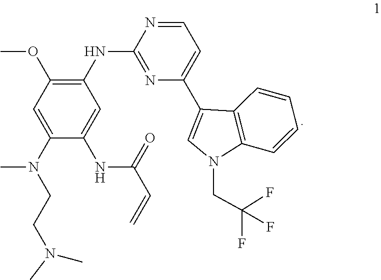 [SMILES: C=CC(=O)Nc1cc(Nc2nccc(-c3cn(CC(F)(F)F)c4ccccc34)n2)c(OC)cc1N(C)CCN(C)C]

2. The salt of claim 1, which is the p-toluenesulfonate of Compound 1 in crystalline form I having the following characteristics: the molar ratio of Compound 1 to p-toluenesulfonic acid is about 1:1, and (a) its X-ray powder diffraction pattern has one or more (1, 2, 3, 4, 5, or 6) peaks at 7.22, 7.90, 9.30, 10.46, 14.64, 15.36, ±0.2° 2θ; and/or (b) its DSC graph has an endothermic peak with an onset temperature of 161.54° C. ±5° C.;
preferably, the X-ray powder diffraction pattern of the crystalline form I of p-toluenesulfonate of Compound 1 has 6 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 7.221 | 13.478 | 17.536 | 20.498 | 23.679 | 28.449 |
| 7.904 | 14.638 | 18.385 | 21.368 | 24.457 | 29.728 |
| 9.293 | 15.36 | 19.004 | 22.224 | 25.408 | 30.176 |
| 10.459 | 15.708 | 19.25 | 22.529 | 26.66 | 31.107 |
| 12.015 | 16.892 | 20.231 | 23.184 | 27.37 | |

preferably, the crystalline form I of p-toluenesulfonate of Compound 1 shows an X-ray powder diffraction pattern substantially the same as that in FIG. 14; preferably, the crystalline form also shows a DSC graph substantially the same as that in FIG. 15.

3. The salt of claim 1, which is the benzenesulfonate of Compound 1 in crystalline form I having the following characteristics: the molar ratio of Compound 1 to benzenesulfonic acid is about 1:1, and (a) its X-ray powder diffraction pattern has one or more (1, 2, 3, 4, or 5, preferably 5) peaks at 8.41, 16.53, 18.78, 21.18, 23.16, ±0.2°, 2θ; and/or (b) its DSC graph has an endothermic peak with an onset temperature of 155.49° C. ±5° C.;
preferably, the X-ray powder diffraction pattern of the crystalline form I of benzenesulfonate of Compound 1 has 6 or more (such as 10, 16, or 20) X-ray diffraction peaks as shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 7.675 | 13.3 | 17.122 | 21.177 | 24.769 | 30.277 |
| 8.411 | 14.595 | 17.728 | 21.532 | 25.162 | 33.549 |
| 10.009 | 15.523 | 18.196 | 22.191 | 25.846 | 34.355 |
| 10.494 | 15.89 | 18.782 | 23.163 | 26.396 | 34.441 |
| 10.766 | 16.534 | 19.181 | 24.082 | 27.523 | 39.824 |
| 11.143 | 16.845 | 20.084 | 24.415 | 29.625 | |

more preferably, the X-ray powder diffraction pattern has diffraction peaks at 7.68, 8.41, 14.60, 15.52, 16.53, 16.85, 17.73, 18.78, 20.08, 21.18, 23.16, 24.42, and 24.76, ±0.2° 2θ;
preferably, the crystalline form I of benzenesulfonate of Compound 1 shows an X-ray powder diffraction pattern substantially the same as that in FIG. 19, preferably, the crystalline form also shows a DSC graph substantially the same as that in FIG. 20.

4. The salt of claim 1, which is the succinate of Compound 1, which is in crystalline form I having the following characteristics: the molar ratio of Compound 1 to succinic acid is about 1:1 and (a) its X-ray powder diffraction pattern has one or more (1, 2, 3, 4, or 5, preferably 5) peaks at 7.38, 10.21, 11.59, 17.55, 23.38, ±0.2° 2θ; and/or (b) its DSC graph has an endothermic peak with an onset temperature of 108.3° C. ±5° C.;
preferably, the X-ray powder diffraction pattern of the crystalline form I of succinate of Compound 1 has 6 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.946 | 13.549 | 17.811 | 21.142 | 25.463 | 29.9 |
| 7.376 | 13.952 | 18.449 | 21.864 | 25.892 | 30.547 |
| 9.175 | 14.89 | 18.642 | 22.144 | 26.463 | 31.357 |
| 9.674 | 15.942 | 19.051 | 23.376 | 27.119 | 31.958 |
| 10.209 | 16.57 | 19.42 | 24.111 | 27.829 | 33.223 |
| 10.672 | 16.859 | 19.595 | 24.402 | 28.567 | 35.668 |
| 11.594 | 17.554 | 20.418 | 24.975 | 29.326 | 36.201 |

more preferably, the X-ray powder diffraction pattern has diffraction peaks at 7.38, 9.18, 9.67, 10.21, 10.67, 11.59, 13.55, 14.89, 16.86, 17.55, 19.05, 19.42, 19.60, 23.38, 24.11, 24.40, 27.83, 29.90, and 30.55, ±0.2° 2θ;
preferably, the crystalline form I of succinate of Compound 1 shows an X-ray powder diffraction pattern substantially the same as that in FIG. 24; preferably, the crystalline form also shows a DSC graph substantially the same as that in FIG. 25; or,
the succinate of Compound 1 is in crystalline form II having the following characteristics: the molar ratio of Compound 1 to succinic acid is about 1:1, and (a) its X-ray powder diffraction pattern has one or more (1, 2, 3, 4, 5, 6, 7, or 8, preferably 5 or more, more preferably, 8) peaks at 7.32, 9.02, 9.65, 10.09, 11.63, 17.53, 19.47, 23.45, ±0.2° 2θ; and/or (b) its DSC graph has an endothermic peak with an onset temperature of 139.9° C. ±5° C.;
preferably, the X-ray powder diffraction pattern of the crystalline form II of succinate of Compound 1 has 8 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.89 | 13.881 | 18.644 | 22.561 | 25.942 | 30.688 |
| 7.321 | 14.734 | 18.945 | 23.148 | 26.482 | 31.826 |
| 8.014 | 15.781 | 19.474 | 23.454 | 26.897 | 33.307 |
| 9.022 | 16.446 | 19.702 | 23.786 | 27.402 | 34.561 |
| 9.652 | 16.774 | 20.376 | 24.171 | 28.108 | 35.276 |
| 10.087 | 17.534 | 21.106 | 24.428 | 29.431 | 36.167 |
| 10.51 | 17.821 | 21.8 | 24.839 | 29.892 | 36.427 |
| 11.63 | 18.131 | 22.293 | 25.349 | 30.33 | 39.608 |
| 13.604 | | | | | |

more preferably, the X-ray powder diffraction pattern has diffraction peaks at 7.32, 9.02, 9.65, 10.09, 10.51, 11.63, 13.60, 14.73, 16.45, 16.77, 17.53, 18.13, 19.47, 19.70, 23.45, 23.79, and 24.43, ±0.2° 2θ;
preferably, the crystalline form II of succinate of Compound 1 shows an X-ray powder diffraction pattern substantially the same as that in FIG. 29; preferably, the crystalline form also shows a DSC graph substantially the same as that in FIG. 30.

5. The salt of claim 1, which is the hydrochloride of Compound 1 in crystalline form III having the following characteristics: the molar ratio of Compound 1 to hydrochloric acid is about 1:1, and (a) its X-ray powder diffraction pattern has one or more (1, 2, 3, 4, 5, 6, 7, or 8, preferably 5 or more, more preferably, 8) peaks at 6.39, 7.35, 10.03, 11.48, 15.27, 21.04, 21.87, 23.35, 24.94, ±0.2° 2θ; and/or (b) its DSC graph has an endothermic peak with an onset temperature of 270.75° C. ±5° C.;
preferably, the X-ray powder diffraction pattern of the crystalline form III of hydrochloride of Compound 1 has 8 or more (such as 10, 16, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.385 | 13.255 | 19.4 | 22.134 | 26.206 | 29.921 |
| 7.353 | 14.632 | 20.042 | 22.745 | 26.789 | 31.559 |
| 7.872 | 15.266 | 20.313 | 23.353 | 27.255 | 32.794 |
| 10.033 | 15.657 | 20.694 | 23.621 | 27.481 | 33.388 |
| 11.483 | 16.947 | 21.037 | 24.101 | 27.875 | 37.271 |
| 12.445 | 18.181 | 21.485 | 24.944 | 28.937 | 39.086 |
| 12.977 | 18.713 | 21.867 | | | |

more preferably, its X-ray powder diffraction pattern has diffraction peaks at 6.39, 7.35, 7.87, 10.03, 11.48, 15.27, 21.04, 21.87, 22.13, 22.74, 23.35, 24.94 and 26.79, ±0.2° 2θ;
preferably, the crystalline form III of hydrochloride of Compound 1 shows an X-ray powder diffraction pattern substantially the same as that in FIG. 4, preferably, the crystalline form also shows a DSC graph substantially the same as that in 5.

6. The salt of claim 1, which is the phosphate of Compound 1 in crystalline form I having the following characteristics: the molar ratio of Compound 1 to phosphoric acid is about 1:1, and (a) its X-ray powder diffraction pattern has one or two (preferably 2) peaks at 8.14, 16.32, ±0.2° 2θ; and/or (b) its DSC graph has an endothermic peak with an onset temperature of 234.95° C. ±5° C.;
preferably, the X-ray powder diffraction pattern of the crystalline form I of phosphate of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 8.144 | 13.554 | 17.395 | 20.994 | 24.015 | 29.882 |
| 8.573 | 14.334 | 17.752 | 21.366 | 24.715 | 31.536 |
| 9.48 | 14.767 | 18.48 | 22.361 | 26.218 | 32.976 |
| 10.988 | 15.671 | 19.362 | 22.992 | 26.91 | 37.285 |
| 12.698 | 16.316 | 20.389 | 23.451 | 29.013 | 39.543 |

more preferably, its X-ray powder diffraction pattern has diffraction peaks at 8.14, 16.32, 17.75 and 20.99, ±0.2° 2θ;
preferably, the crystalline form I of phosphate of Compound 1 shows an X-ray powder diffraction pattern substantially the same as that in FIG. 9; preferably, the crystalline form also shows a DSC graph substantially the same as that in FIG. 10.

7. The salt of claim 1, which is the sulfate of Compound 1 in crystalline form I having the following characteristics: the molar ratio of Compound 1 to sulfuric acid is about 1:1 and (a)its X-ray powder diffraction pattern has one or more (preferably 2 or 3) peaks at 10.28, 18.34, 20.64, ±0.2° 2θ; and/or (b) its DSC graph has an endothermic peak with an onset temperature of 255.89° C. ±5° C.;
preferably, the X-ray powder diffraction pattern of the crystalline form I of sulfate of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 9.039 | 14.239 | 20.141 | 21.943 | 27.196 | 31.141 |
| 9.49 | 15.432 | 20.411 | 22.45 | 28.534 | 32.097 |
| 10.275 | 18.342 | 20.635 | 22.792 | 30.647 | 33.216 |
| 11.809 | 19.085 | 21.261 | 24.479 | | |

more preferably, its X-ray powder diffraction pattern has diffraction peaks at 9.04, 10.28, 18.34, 20.41, 20.64, 27.20 and 28.53, ±0.2° 2θ;
preferably, the crystalline form I of sulfate of Compound 1 shows an X-ray powder diffraction pattern substantially the same as that in FIG. 32, preferably, the crystalline form also shows a DSC graph substantially the same as that in FIG. 33.

8. The salt of claim 1, which is the hydrobromide of Compound 1, wherein the hydrobromide salt is a monohydrobromide salt in crystalline form I having the following characteristics: the molar ratio of Compound 1 to hydrobromic acid is about 1:1, and (a) its X-ray powder diffraction pattern has one or two peaks at 6.10, 24.73 ±0.2° 2θ; and/or (b) its DSC graph has two endothermic peaks;
preferably, the X-ray powder diffraction pattern of the crystalline form I of monohydrobromide of Compound 1 has 4 or more (such as 6, 10, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 3.67 | 13.07 | 17.703 | 23.634 | 28.981 | 31.923 |
| 6.104 | 14.58 | 19.27 | 24.73 | 29.532 | 37.951 |
| 10.262 | 15.651 | 20.057 | 26.032 | 30.584 | 39.358 |
| 12.251 | 16.739 | 21.916 | 26.437 | 31.816 | |

more preferably, its X-ray powder diffraction pattern has diffraction peaks at 6.10, 12.25, 13.07, 14.58, 15.65, 16.74, 19.27, 20.06, 21.92, 24.73, 26.03 and 26.44, ±0.2° 2θ;
preferably, the crystalline form I of monohydrobromide of Compound 1 shows an X-ray powder diffraction pattern substantially the same as that in FIG. 37; preferably, the crystalline form also shows a DSC graph substantially the same as that in FIG. 38; or,
the hydrobromide of Compound 1 is a dihydrobromide in crystalline form I having the following characteristics: the molar ratio of Compound 1 to hydrobromic acid is about 1:2, and (a) its X-ray powder diffraction pattern has one or more (such as 1, 2, 3, or 4) peaks at 6.28, 13.12, 19.30, 25.34, ±0.2° 2θ; and/or (b) its DSC graph has two endothermic peaks with onset temperatures at 193.38° C. ±5° C. and 230.24° C. ±5° C. respectively;
preferably, the X-ray powder diffraction pattern of the crystalline form I of dihydrobromide of Compound 1 has 6 or more (such as 8, 12, or 20) X-ray diffraction peaks shown in the table below:

| Angle | Angle | Angle | Angle | Angle | Angle |
|---|---|---|---|---|---|
| 2θ/° | 2θ/° | 2θ/° | 2θ/° | 2θ/° | 2θ/° |
| 6.276 | 12.071 | 18.953 | 22.87 | 28.58 | 33.849 |
| 7.329 | 12.603 | 19.305 | 23.626 | 29.384 | 34.543 |
| 7.771 | 13.122 | 19.605 | 24.148 | 30.618 | 35.211 |
| 9.38 | 14.575 | 20.387 | 25.341 | 31.164 | 36.629 |
| 9.69 | 16.777 | 20.662 | 25.61 | 31.832 | 38.6 |
| 10.493 | 17.067 | 21.148 | 26.424 | 32.348 | 39.414 |
| 11.591 | 18.236 | 21.954 | 27.78 | 33.126 | |

more preferably, its X-ray powder diffraction pattern has diffraction peaks at 6.28, 13.12, 16.78, 18.95, 19.30, 21.95, 23.63, 25.34, 25.61 and 26.42, ±0.2° 2θ;
preferably, the crystalline form I of dihydrobromide of Compound 1 shows an X-ray powder diffraction pattern substantially the same as that in FIG. 40, preferably, the crystalline form also shows a DSC graph substantially the same as that in FIG. 41.

9. A pharmaceutical composition comprising the salt of Compound 1 according to claim 1 and a pharmaceutically acceptable carrier or diluent.

10. Use of the salt of claim 1 in the preparation of a drug for the treatment or prevention of a disorder or disease mediated by activating or resistant mutant form of EGFR, for example, mediated by L858R activating mutant, Exon19 deletion activating mutant and/or T790M resistance mutant of EGFR;
preferably, the disorder or disease is selected from one or more selected from one or more of the following: ovarian cancer, cervical cancer, colorectal cancer (e.g., colon adenocarcinoma), breast cancer, pancreatic cancer, glioma, glioblastoma, melanoma, prostate cancer, leukemia, lymphoma, non-Hodgkin's lymphoma, gastric cancer, lung cancer (for example, non-small cell lung cancer), hepatocellular carcinoma, gastrointestinal stromal tumor (GIST), thyroid cancer, cholangiocarcinoma, intrauterine membrane cancer, kidney cancer, anaplastic large cell lymphoma, acute myeloid leukemia (AML), multiple myeloma or mesothelioma.
